(19) United States
(12) Patent Application Publication
(10) Pub. No.: US 2007/0128116 A1
(43) Pub. Date: Jun. 7, 2007

(54) MULTIFUNCTIONAL CORE FOR MOLECULAR IMAGING AND TARGETED DELIVERY OF MACROMOLECULES AND DRUGS

(76) Inventors: Zheng J. Wang, San Antonio, TX (US); Irene Kazhdan, San Antonio, TX (US); Jian Gao, Helotes, TX (US); Zhiqiang Han, San Antonio, MO (US); Robert A. Marciniak, San Antonio, TX (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/460,500

(22) Filed: Jul. 27, 2006

Related U.S. Application Data
(60) Provisional application No. 60/702,713, filed on Jul. 27, 2005.

(51) Int. Cl. A61K 49/00; A61K 48/00; A61K 31/409; A61K 31/555

(52) U.S. Cl. 424/9.6; 514/44; 424/9.61; 514/185; 514/410

(57) ABSTRACT

In the broadest sense an inventive conjugate includes a macrocyclic scaffold moiety incorporating at least one nitrogen atom. Through linkages to the ring, nitrogen atoms are bonded to at least one therapeutic species, or one cellular trafficking species, and preferably two or more types of trafficking species. The linkages between ring nitrogen atoms and agents are formed through N—C, N—S, N—O or N—N bonds. It is appreciated that more than one agent or agent species are coupled to a ring nitrogen atom through the inclusion of a branched carbon atom or secondary amine nitrogen within the intermediate between the agent and ring nitrogen atom. A disulfide bond intermediate between an agent and a linkage to the ring nitrogen is preferred in instances where intracellular release of the agent or trafficking species is desired. An optional metal ion is coordinated by the scaffolding moiety.

### RELATED APPLICATIONS

This application is a non-provisional application that claims priority benefit to U.S. Provisional Application Ser. No. 60/702,713 filed Jul. 27, 2005; the contents of which are hereby incorporated by reference,

### GRANT INFORMATION

The subject invention was made with the support of a grant from the San Antonio Life Sciences Institute, Grant No. 00000010001377. The Institute may have certain rights in the invention.

### FIELD OF THE INVENTION

The invention relates to compositions and methods for targeted delivery of a molecule. In particular, the invention relates to compositions and methods for delivery of a molecule to a cell or tissue of an organism.

### BACKGROUND OF THE INVENTION

Various methods and compositions are used as delivery vehicles for macromolecules such as DNA, RNA, proteins, oligonucleotides and peptides in addition to small molecule therapeutics. Numerous difficulties have been encountered in delivering such molecules to a desired target including poor penetration into a tissue or cell, toxicity when delivered systemically due to insufficient specificity of targeting to a particular tissue or cell, side effects when delivered in high concentration in order to achieve an adequate local concentration at a particular target cell or tissue, and degradation such that inadequate amounts are delivered to the target and/or such that byproducts of degradation result in undesirable side effects.

Numerous vehicles have been generated for the purpose of molecular cargo delivery including viral vectors, liposomes and niosomes. However, these various vectors have their limitations. For example, various viral vectors work well in vitro but have low infection rates in many tissues. Further, many viral vectors have the additional drawback of generating an immune response. In addition, many such delivery systems are limited by their ability to cross cellular and nuclear membranes. Even where such compositions do cross cell membranes, they are often limited in their efficacy by entrapment in endosomes, limiting their functional abilities.

Thus, there is a continuing need for methods and compositions for delivery of a molecular cargo to a specified cell or tissue.

### BRIEF DESCRIPTION OF THE DRAWINGS

FIG. 1A is a drawing schematically illustrating an embodiment of an inventive conjugate including a “cyclen” scaffold specie.

FIG. 1B is a drawing schematically illustrating an embodiment of an inventive conjugate including a “cyclen” scaffold specie.

FIG. 2 is a drawing illustrating chemical modification of a tetrazamacrocycle.

FIG. 3 is a drawing illustrating conjugation of two translocating species to a scaffold specie.

FIG. 4 is a drawing illustrating modification of an oligonucleotide in preparation for conjugation to a scaffold specie.

FIG. 5 is a drawing illustrating addition of an oligonucleotide to a Tat-scaffold conjugate.

FIG. 6 is a drawing illustrating two scaffold species incorporated in selected embodiments of an inventive conjugate.

FIG. 7A is a reproduction of a digital image illustrating SK-BR-3 cells incubated with FITC labeled TC (TC-FITC) examined by confocal microscopy.

FIG. 7B is graph showing flow cytometry data following incubation of cells with a TC-FITC conjugate.

FIG. 8 is a graph illustrating quantitation of uptake of FITC-labeled TC conjugates into SK-BR-3 and HCC1500 cells FIG. 9A is a reproduction of a digital image illustrating distribution of fluorescently labeled Tat-CYCLEN antisense oligonucleotide conjugates (TC-1-TAMR-ASON) in SK-BR-3 cells.

FIG. 9B is a reproduction of a digital image illustrating distribution of fluorescently labeled Tat-CYCLEN antisense oligonucleotide conjugates (TC-1-TAMR-ASON) in SK-BR-3 cells.

FIG. 10A is a reproduction of a digital image illustrating distribution of fluorescently labeled Tat-CYCLEN antisense oligonucleotide conjugates (TC-1-TAMR-ASON) in SK-BR-3 cells.

FIG. 10B is a reproduction of a digital image illustrating distribution of FM1-43 in SK-BR-3 cells.

FIG. 10C is a reproduction of a digital image illustrating overlapping signal from TC-1-TAMR-ASON conjugates and FM1-43 in SK-BR-3 cells.

FIG. 10D is a reproduction of a light microscopic image of the SK-BR-3 cells in FIGS. 10A-C.

FIG. 11 is a schematic illustration of synthesis of a radiolabeled conjugate.

FIG. 12 is a graph illustrating uptake of a radiolabeled Tat-CYCLEN conjugate into cultured cells.

FIG. 13 is a graph illustrating cell uptake of FITC-labeled conjugates having one or two translocating moieties.

FIG. 14 is a graph illustrating cell uptake of radiolabeled conjugates having one or two translocating moieties.

FIG. 15A-A is a reproduction of a confocal micrograph illustrating in vitro delivery of an oligonucleotide conjugate.

FIG. 15A-B is a reproduction of a micrograph illustrating in vivo delivery of a radiolabeled aptamer conjugate.

FIG. 15B is graph depicting cell penetration of an inventive conjugate in conjunction with a reproduction of a fluorescence micrograph of tissue after inventive conjugate uptake.

FIGS. 16A, C, E, and G show GPI-specific autoantibodies localized to distal joints of normal mice.

FIGS. 16D, F and H show much lower levels of anti-GPI in distal joints of three types of knockout mouse, FcR knockout, TNF-α knockout and C3 knockout respectively.

FIG. 16B shows distribution of normal IgG in a control mouse.

FIG. 17A is a graph illustrating microPET digital scanning data of ⁶⁴Cu radioactivity density in the ankle region of a C5+ mouse and a C5 knockout mouse.

FIG. 17B is a graph illustrating microPET digital scanning data of ⁶⁴Cu radioactivity density in the ankle region of a wild-type control mouse compared to that of an FcR knockout mouse.

FIG. 18A is a schematic illustration of synthesis of a conjugate including an siRNA and three Tat translocating molecules.

FIG. 19 is a schematic illustration of synthesis of a radiolabeled conjugate including an antibody.

FIG. 20 is a drawing illustrating a synthetic scheme for modification of arginine to include a photoactive linker.

FIG. 21 is a drawing illustrating a scheme for synthesis of a conjugate including a photoactive linker,

FIG. 22 is a drawing illustrating a scheme for synthesis of a conjugate including a Tat moiety and an endosome disruption moiety along with a photoactive linker,

FIG. 23 is a drawing illustrating a scheme for synthesis of a conjugate including a Tat moiety and an endosome disruption moiety along with a photoactive linker and an oligonucleotide.

FIG. 24 is a drawing illustrating a scheme for synthesis of a conjugate including a Tat moiety, an endosome disruption, an oligonucleotide and an antibody.

### SUMMARY OF THE INVENTION

In the broadest sense an inventive conjugate includes a macrocyclic scaffold moiety incorporating at least one nitrogen atom. Through linkages to the ring, nitrogen atoms are bonded to at least one therapeutic species, or one cellular trafficking species, and preferably two or more types of trafficking species. The linkages between ring nitrogen atoms and agents are formed through N—C, N—S, N—O or N—N bonds, It is appreciated that more than one agent or agent species are coupled to a ring nitrogen atom through the inclusion of a branched carbon atom or secondary amine nitrogen within the intermediate between the agent and ring nitrogen atom. A disulfide bond intermediate between an agent and a linkage to the ring nitrogen is preferred in instances where intracellular release of the agent or trafficking species is desired. An optional metal ion is coordinated by the scaffolding moiety.

A variety of medical conditions and diagnostics are performed by delivery of an inventive conjugate as part of a pharmaceutical composition.

### DETAILED DESCRIPTION OF THE INVENTION

Inventive conjugates and methods have utility for delivery of a molecular cargo to a specified cell or tissue. An inventive conjugate includes a scaffold moiety and one or more cargo moieties and one or more trafficking species attached to the scaffold specie.

The term “scaffold specie” as used herein refers to an organic macrocycle which serves as a support for one or more cargo species which are covalently coupled to the scaffold specie. In a preferred option, a scaffold specie is an organic macrocycle chelant. The term “covalently coupled” as used herein is intended to mean that the coupled moieties are directly bonded to each other, or indirectly bonded to each other, such as by a linker.

The term “trafficking specie” as used herein refers to a functional molecule attached to a scaffold specie. A trafficking molecule includes a molecule functional to aid in delivery of an inventive conjugate to a cell or a subcellular location such as an organelle or the cytoplasm. Thus, exemplary trafficking molecules include a cellular targeting moiety, a translocating moiety, an endosome disruption moiety, and an organelle targeting moiety.

The term “cargo specie” as used herein refers to therapeutic or diagnostic RNA, DNA, a peptide, drug, prodrug or reporter that is transported by an inventive conjugate into proximity to a target cell or tissue.

An inventive conjugate has the general form 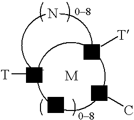
of an azamacrocycle is provided as a scaffolding specie including at least three nitrogen atoms separated by alkyl, aryl, thioether, or ether coupling groups. The scaffolding specie has up to eleven nitrogen atoms and an optional second cycle so as to form a cryptand. At least three nitrogen atoms are bonded to agents including a first type of trafficking specie that is a cellular targeting moiety, a translocating moiety, an endosome disruption moiety and an organelle targeting moiety; at least a second type of trafficking specie distinction from the first type of trafficking agent; and at least a cargo specie having therapeutic or report effect.

The term “organic macrocycle” as used herein refers to a heterocycle structure containing about seven to sixty atoms linked to form a ring backbone. The ring backbone atoms include carbon, nitrogen, oxygen, sulfur, and/or phosphorus, for instance.

Organic macrocycles illustratively include, without limitation, polyazamacrocycles, crown ethers, oxa-aza crown ether, thia-aza crown ethers, thia-oxa crown ethers, aza-thia-oxa crown ethers, thia crown ethers, cryptands, cavitands, podands, calixarenes, cyclophanes, corrinoids, porphyrins, benzophyrins, porphycenes, chlorins, benzochlorins and purpurins. Organic macrocycles are described in references including Gerbeleu, N. V. et al., Template Synthesis of Macrocyclic Compounds, Wiley Pub., 1999; PCT International Application W09403464; Alexander, V., Chem. Rev. 95:273-342, 1995; and D. Parker (Ed.), Macrocycle Synthesis: A Practical Approach, Oxford University Press, 1996.

In one embodiment, an organic macrocycle included in an inventive conjugate is an organic macrocycle chelant.

The term “chelant” as used herein refers to a molecule containing two or more electron donor atoms capable of chelating or sequestering an ion, particularly a metal ion, such as by forming a coordination bond with the ion. The term “chelate” refers to the structure that is formed by the chelant and ion. Organic macrocycle chelants are organic macrocycles capable of chelating or sequestering an ion, particularly a metal ion, such as by forming a coordination bond with the ion. Such chelants are known in the art and include specific polyazamacrocycles, crown ethers, oxa-aza crown ether, thia-aza crown ethers, thia-oxa crown ethers, aza-thia-oxa crown ethers, thia crown ethers, cryptands, cavitands, podands, calixarenes, cyclophanes, corrinoids, porphyrins, benzophyrins, porphycenes, chlorins, benzochlorins and purpurins.

It is appreciated that multiple scaffold species are preferred to form an inventive conjugate for delivery of a high molecular weight cargo specie, such as a plasmid DNA.

For delivery of DNA, multiple scaffold specie cores are attached to one plasmid DNA. This strategy allows for the delivery of large molecular weight DNA. To facilitate DNA delivery in a first embodiment, the DNA is modified with amine groups at the phosphate sites and then undergoes conjugation with the scaffold specie cores via vinyl moieties. Alternatively, DNA is first packaged with a DNA-compacting peptide. A number of compacting peptides (also called packaging peptides) have been described in the literature, including short stretches of poly-1-lysine, protamine, etc. A representative packaging peptide operative herein is CGKKKFKLKH-CONH₂. After DNA is packaged, multiple scaffolding specie cores are attached to the peptides in the DNA-packaging peptide complex. Another order of construction using packing peptides covalently attaches a packaging peptide to a scaffold specie of a multiple scaffolding specie core, and thereafter DNA is introduced to induce DNA packaging around the packaging peptides.

An organic macrocycle scaffold included in an inventive conjugate is a polyazamacrocycle having the formula: 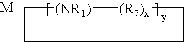
where each R₁ is independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group; where each R₇ is independently a C₁-C₁₀ straight chain or branched, saturated or unsaturated, substituted or unsubstituted alkyl, or aryl group; where each x is an integer independently chosen from the range of 1-4, inclusive; where y is an integer in the range of 2-8, inclusive; and where M is an optional metal ion.

In another embodiment, an organic macrocycle scaffold included in an inventive conjugate is a polyazamacrocycle having the formula: 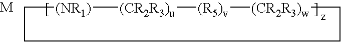
where each R₁, R₂ and R₃ is independently in each occurrence H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ allcyl or aryl group; where z is an integer in the range of 2-8, inclusive; where each u is an integer independently chosen in each occurrence from the range of 1-4, inclusive; where R₅ is hydroxy, carboxy, amine, halogen, nitrogen moiety, sulfur moiety, phosphorus moiety, oxygen moiety, a C₁-C₁₀ straight chain or branched, saturated or unsaturated, substituted or unsubstituted alkyl, or aryl group; where each v is an integer independently chosen from 0 or 1; where each w is an integer independently chosen in each occurrence from the range of 0-4, inclusive; and where M is an optional metal ion.

In one option, R₁, R₂ and R₃ are H in each instance.

In still another embodiment, an organic macrocycle included in an inventive conjugate is a polyazamacrocycle having the formula: 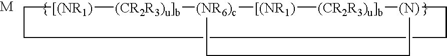
where each R₁, R₂ and R₃ is independently in each occurrence H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group; where y is an integer in the range of 2-8, inclusive; where each x is an integer independently chosen in each occurrence from the range of 1-4, inclusive; where R₆ is a C₁-C₁₀ straight chain or branched, saturated or unsaturated, substituted or unsubstituted alkyl, or aryl group; where b is an integer in the range of 2-8, inclusive; where each u is an integer independently chosen in each occurrence from the range of 1-4, inclusive; where each c is an integer independently chosen in each occurrence from 0 or 1; and where M is an optional metal ion.

In a one option, R₆ is —(CH₂)₂(NR₁)(CH₂)₂NR₁)(CH₂)₂—. In a further option, R₁, R₂ and R₃ are H in each instance.

In a further embodiment, a scaffold macrocycle has the formula: 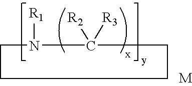
where each R₁ is independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group, or a combination thereof; where each R₂ and R₃ are independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group; where y is an integer in the range of 2-8, inclusive; where each u is an integer independently chosen from the range of 1-4, inclusive; and where M is an optional metal ion.

As noted, M is an optional metal ion associated with an inventive conjugate by coordination bonding. In one embodiment, M is a lanthanide, transition metal or heavy metal. Optionally, M is a radionuclide useful in imaging and/or as a therapeutic as described herein.

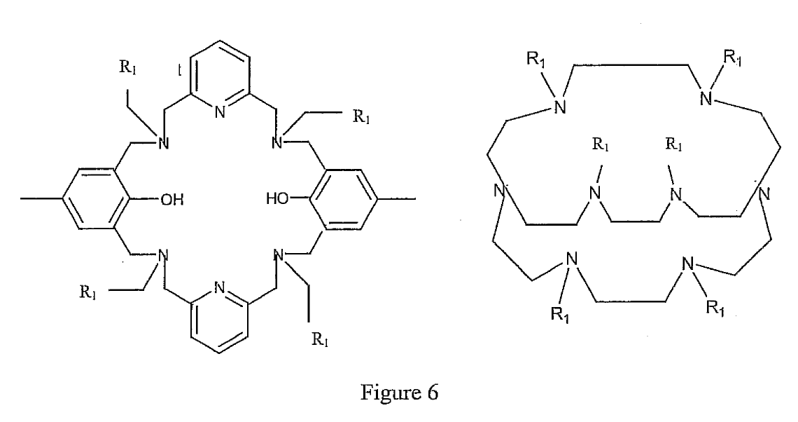

In a particular embodiment, a polyazamacrocycle included in an inventive conjugate includes a cyclam or a cyclen tetrazamacrocycle. Further examples of particular polyazamacrocycles included in an inventive conjugate are shown in FIG. 6.

In still a further embodiment, a scaffold macrocycle has a formula 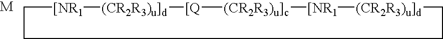
where each R₁ is independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group, or a combination thereof; where each R₂ and R₃ are independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group; where Q is independently in each occurrence oxygen or sulfur; where each u is an integer independently chosen from the range of 1-4, inclusive; where d is an integer independently chosen from the range of 1-4, inclusive; e is an integer independently chosen from the range of 1-3, inclusive; and where M is an optional metal ion.

A scaffold specie as described herein may be obtained commercially or synthesized by methods known in the art such as detailed in references as exemplified in Gerbeleu, N. V. et al., Template Synthesis of Macrocyclic Compounds, Wiley Pub., 1999; PCT International Application W09403464; Alexander, V., Chem. Rev. 95:273-342, 1995; and D. Parker (Ed.), Macrocycle Synthesis: A Practical Approach, Oxford University Press, 1996, chapters 1-4; and Blake, A. J. et al., Acta Cryst. E60:0901-0903, 2004.

Conjugates

As noted above, an inventive conjugate includes a scaffold specie, one or more cargo species, and one or more trafficking species attached to the scaffold specie. It is appreciated that multiple scaffold species can be incorporated into an inventive conjugate, the scaffold species being coupled together, or separated by cargo or trafficking species.

In one embodiment, a conjugate composition includes an azamacrocycle scaffold conjugated to a trafficking specie that is a targeting moiety, a reporter moiety, a translocating moiety, an endosome disruption moiety, or combinations thereof; or a cargo specie that is a therapeutic or diagnostic DNA, RNA, protein, drug, prodrug or marker.

In a further embodiment, a conjugate composition includes an azamacrocycle scaffold conjugated to a therapeutic or diagnostic moiety, a targeting moiety, a translocating moiety, and combinations thereof. A reporter moiety and/or a second translocating moiety, a second endosome disruption moiety, and/or a second therapeutic moiety are optionally included.

In another embodiment, a conjugate composition includes an azamacrocycle scaffold conjugated to a therapeutic or diagnostic moiety, a cellular targeting moiety, an endosome disruption moiety, an organelle targeting moiety, and combinations thereof. A reporter moiety and/or one or more translocating moieties, a second endosome disruption moiety, a second targeting moiety and/or a second therapeutic moiety are optionally included.

In a further embodiment, three or more translocating moieties, endosome disruption moieties, reporter moieties, targeting moieties or therapeutic moieties are optionally included.

In a preferred embodiment, the cargo specie or species are conjugated to the scaffold specie by linkage to a nitrogen member of the azamacrocycle.

In one embodiment, an inventive conjugate has the formula: 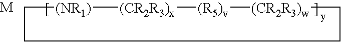
where each R₁ is independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group, a cargo specie, or a moiety LR₄, where R₄ is a cargo specie, and where L is a linker moiety; where at least one moiety R₁ is a cargo specie or a moiety LR₄ is present; where each R₂ and R₃ are independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group, a cargo moiety, or a moiety LR₄ wherein R₄ is a cargo moiety, and where L is a linker moiety; where y is an integer in the range of 2-8, inclusive; where each x is an integer independently chosen from the range of 1-4, inclusive; where R₅ is a C₁-C₈ straight chain or branched, substituted or unsubstituted alkyl, alkenyl or arylalkyl; where each v is an integer independently chosen from 0 or 1; where each w is an integer independently chosen from the range of 0-4, inclusive; and where M is an optional metal ion.

In a preferred option, R₂ and R₃ are H in each instance.

In one embodiment, an organic macrocycle chelant included in an inventive conjugate is a polyazamacrocycle having the formula: 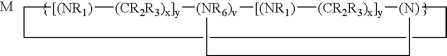
where each R₁ is independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group, a cargo specie or a moiety LR₄, where R₄ is a cargo specie and where L is a linker moiety; where at least one moiety R₁ is a cargo specie or a species LR₄ is present; where each R₂ and R₃, are independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group, a cargo specie or a moiety LR₄ wherein R₄ is a cargo specie, and where L is a linker moiety; where y is an integer in the range of 2-8, inclusive; where each x is an integer independently chosen from the range of 1-4, inclusive; where R₆ is a C₁-C₈ straight chain or branched, substituted or unsubstituted alkyl, alkenyl; where each v is an integer independently chosen from 0 or 1; where each w is an integer independently chosen from the range of 0-4, inclusive; and where M is an optional metal ion.

In a preferred option, R₆ is —(CH₂)₂(NR₁)(CH₂)₂(NR₁)(CH₂)₂—.

In another embodiment, an inventive conjugate has the formula: 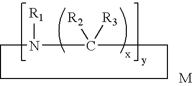
where each R₁ is independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group, a trafficking specie, a cargo specie, a combination thereof, or a moiety LR₄, where R₄ is a cargo specie and where L is a linker moiety; where at least one moiety R₁ is a cargo specie or a moiety LR₄ is present; where each R₂ and R₃ are independently H, hydroxy, carboxy, amine, halogen, nitrogen moiety, sulfur moiety, phosphorus moiety, oxygen moiety, a straight chain or branched, saturated or unsaturated, substituted or unsubstituted C₁-C₁₀ alkyl or aryl group, a cargo moiety, and where L is a linker moiety; where y is an integer in the range of 2-8, inclusive; where each x is an integer independently chosen from the range of 1-4, inclusive; and where M is an optional metal ion.
Conjugation of a Trafficking or Cargo Specie to a Scaffold

A trafficking or cargo specie is conjugated directly to a scaffold specie or indirectly, via a linker.

The term “linker” as used herein refers to molecules joining a scaffold specie and a trafficking or cargo specie, preferably covalently.

In one embodiment, broadly described, a linker is a hetero- or homo-bifunctional moiety that includes a reactive functional group for reaction with a reactive functional group on the scaffold specie to form a linkage between the linker and the scaffold specie. A linker generally further includes a reactive functional group for reaction with a reactive functional group on the cargo moiety to form a linkage between the linker and the cargo moiety.

In addition, the term “linker” also includes covalent coupling of a trafficking or cargo specie and a scaffold specie by addition of a first linker to the scaffold specie and addition of a second linker to the trafficking or cargo specie and further reaction of the two linkers to form a linkage. The term “linkage” as used herein is intended to mean a bond or chemical group formed by chemical reaction between two species such that the species are covalently coupled. Common linkages illustratively include ester, imino, amino, disulfide, thiosemicarbazone, semicarbazone, oxime, hemiacetal, hemiketal, ether, thioether, amide, peptide and combinations thereof. Linkages degraded intracellularly such as disulfides are particularly preferred to release a species upon arrival within a target cell.

Linkers and chemical reactions of linkers to form a linkage are known in the art, as are chemical modifications of moieties to be linked to provide them with reactive functional groups if necessary Common reactive functional groups illustratively include carboxyl, hydroxyl, amine, sulfhydryl, phosphate, epoxide, halide, acid chloride, anhydride, acetate, nosylate, brosylate and tosylate.

Exemplary linkers include, for instance and without limitation, spacer molecules such as a straight chain or branched, substituted or unsubstituted, saturated or unsaturated 1-20 carbon alkyl moiety having reactive groups, dendrimers, peptides, proteins, lipids, sugars, polyethylene glycols, oligonucleotides and the like, capable of forming a bond with a scaffold and a cargo moiety such that the cargo moiety is linked to the scaffold. Preferably, a linker is selected such that cargo moiety activity, such as a biological, diagnostic and/or reporter activity of the various cargo molecules, is not significantly inhibited. For example, a linker is selected such that cargo moiety activity is not significantly inhibited by steric interactions with the scaffold or other cargo moieties.

Further exemplary linkers include photoactive coupling agents active to bind a cargo moiety in the presence of a light stimulus, illustratively including p-azidophenyl glyoxal (APG), N-5-azido-2-nitrobenzoylsuccinimide (ANB-NOS), p-azidophenacyl bromide (APB), N-(4-azidophenylthio) phthalimide (APTP), 4,4′-dithio-bis-phenylazide (DTBPA), ethyl 4-azidophenyl-1,4-dithiobutyrimidate (EADB), 4-fluoro-3-nitrophenyl azide (FNPA), N-hydroxysuccinimidyl-4-azidobenzoate (HSAB), N-hydroxysuccinimidyl-4-azidosalicylic acid (NHS-ASA), methyl-4-azidobenzoimidate (MAB), p-nitrophenyl-2-diazo-3,3,3-trifluoropropionate (PNP-DTP), 2-diazo-3,3,3-trifluoropropionyl chloride, N-succinimidyl-6(4′-azido-2′-nitrophenylamino) hexanoate (SANPAH), N-succinimidyl(4-azidophenyl)1,3′-dithiopropionate (SADP), sulfosuccinimidyl-2-(m-azido-o-nitobenzamido)-ethyl-1,3′-dithiopropionate (SAND), sulfosuccinimidyl(4-azidophenyldithio)propionate (Sulfo-SADP), sulfosuccinimidyl-6-(4′-azido2′-nitrophenylamino)hexanoate (Sulfo-SANPAH), sulfosuccinimidyl-2-(p-azidosalicylamido) and ethyl-1,3′-dithiopropionate (SASD)

Materials and methods for linkage formation are known in the art. For example, one method of formation of an amide linkage includes use of a coupling agent such as a carbodiimide. 1-Ethyl-3-(3-dimethylaminopropyl)-carbodiimide (EDC) is an example of water-soluble carbodiimide. Another example of a carbodiimide is the organic soluble compound N,N′-dicyclohexyl-carbodiimide (DCC). Carbodiimide is an example of a coupling agent that catalyzes the formation of amide bonds between carboxylic acids or phosphates and amines. N-Hydroxysuccinimide (NHS) is a reagent that may be included to increase efficiency of carbodiimide mediated coupling reactions.

Optionally, one trafficking or cargo specie is directly bonded to a second trafficking or cargo specie, either directly or through a linker. Thus, for example, a targeting moiety may be bonded to an endosome disruption moiety and/or a translocating moiety may be bonded to a therapeutic moiety. Further, a reporter moiety is illustratively bonded to a targeting moiety. Further permutations are considered to be within the scope of inventive conjugates.

The term “cellular targeting moiety” is intended to mean a moiety functional to enhance delivery of an inventive conjugate to a target. For example, a cellular targeting moiety enhances delivery of a conjugate to a particular target cell type or tissue, such as a cell to which a particular therapeutic treatment is addressed, A cellular targeting moiety illustratively includes a protein, peptide, antibody, antibody fragment, hormone, antigen, hapten, carbohydrate binding moiety such as a lectin, enzyme, enzyme substrate, receptor, receptor ligand, oligonucleotide, aptamer and other such molecules which specifically interact with a binding partner molecule.

In a particular embodiment, a cellular targeting moiety interacts with a marker indicative of a targeted cell. For example, particular tumors express tumor markers indicative of the status of the cell as a tumor. Such markers are known in the art, such as described in M. Fleisher (Ed.), The Clinical Biochemistry of Cancer, Washington, DC: American Association of Clinical Chemists, 1979; R. B. Herbman and D. W. Mercer, eds. hmunodiagnosis of Cancer, 2nd Ed., New York: Marcel Dekker, 1990; and C. T. Garrett, S. Sell (Eds.), Cellular Cancer Markers, Humana Press, 1995. Further cells express cell-type specific markers, such as neurotransmitter receptors, peptide receptors and the like.

The term “antibody” as used herein refers to any of various types of antibody illustratively including a polyclonal, monoclonal, chimeric, humanized and heteroantibody.

The term “antibody fragment” as used herein includes a portion of an antibody such as F(ab′)₂, F(ab)₂, Fab′, Fab, and any portion of an antibody having specificity toward an epitope of interest.

The term “aptarner” as used herein refers to a single-stranded, partially single-stranded, partially double-stranded or double-stranded nucleotide sequence capable of specifically recognizing a selected molecule or a group of molecules such as a protein, peptide, drug, nucleotide, and oligonucleotide. Where the target molecule is a nucleotide or oligonucleotide, an aptamer binds by a non-standard nucleotide-nucleotide binding mechanism, that is, other than Watson-Crick base pairing or triplex formation.

The term “organelle targeting moiety” as used herein refers to a moiety functional to enhance targeting of an inventive conjugate to a particular intracellular location. For example, a nuclear localization signal is a targeting moiety useful in enhancing localization of an inventive conjugate in a cell nucleus. Exemplary nuclear localization sequences are known in the art and are described in references such as Dingwall, C., and Laskey, R. Nuclear targeting sequences—a consensus? 1991, Trends Biochem. Sci. 16:478-481; Mattaj, I., Englmeier. Nucleocytoplasmic transport: the soluble phase. 1998, Annu Rev. Biochem 67:265-306; Gorlich, D., Kutay, U. Transport between the cell nucleus and the cytoplasm. 1999, Annu. Rev. Cell Dev. Biol. 15:607-660; and Nakielny, S., Dreyfiss, G. Transport of proteins and RNAs in and out of the nucleus. 1999, Cell 99:677-690.

The term “therapeutic moiety” as used herein refers to a molecule, group of molecules, complex or substance administered to an organism for diagnostic, therapeutic, and/or medical purposes.

A therapeutic moiety is biologically active compound such as a pharmaceutical, protein, peptide, carbohydrate, lipid, enzyme inhibitor, hormone, cytokine, antigen, bapten, prodrug, polynucleotide, oligonucleotide, nucleic acid, radionuclide, or combination thereof.

The term “biologically active” as used herein refers to a compound which has an effect on a specific biological process. A biologically active compound has activity described at a molecular level such as receptor bindingblocking, receptor activation/inhibition, ion channel modulation and second messenger modulation. Biological activity further includes activity described at a cellular or subcellular level such as stimulation/inhibition of synaptic release. In addition, biological activity further includes activity described at an organismal level such as perceived relief of a symptom or increased organismal activity. In one embodiment, biological activity of a therapeutic compound includes inhibition of growth of a target cell, inhibition of division of a target cell and/or induction or stimulation of death of a target cell, such as a tumor cell. Biological activity of a compound is measurable and may be assessed by techniques known in the art.

A protein or peptide therapeutic moiety which is a therapeutic moiety includes both naturally occurring and non-naturally occurring proteins and peptides. Non-naturally occurring proteins and peptides include those which are not found in an unmodified cell, tissue or organism. A protein or peptide therapeutic moiety includes at least two amino acids linked by a peptide bond and the amino acids so linked may be natural, modified and/or synthetic amino acids.

A protein or peptide therapeutic moiety includes, but is not limited to, an antibody, an antibody fragment, an enzyme, an enzyme inhibitor or modulator, a cytokine, a chemokine, a receptor, a receptor ligand, a hormone, a neurotransmitter, and a neuropeptide.

A therapeutic moiety which is an oligonucleotide includes at least two linked nucleotides and includes molecules having up to about 100 nucleotides. A therapeutic moiety including more than 100 nucleotides is generally termed a polynucleotide. Natural nucleotides contain a deoxyribose or ribose group, a phosphate group, and a base. Bases include purines and pyrimidines, which further include the natural compounds adenine, thymine, guanine, cytosine, uracil, inosine, and natural analogs. Modified or non-natural oligo- and poly-nucleotides include phosphorothioates, phosphorodiamidates, morpholino oligo- and poly-nucleotides.

Synthetic derivatives of purines and pyrimidines include, but are not limited to, modifications which place new reactive groups such as, but not limited to, amines, alcohols, thiols, carboxylates, and alkylhalides. The term “base” encompasses any of the known base analogs of DNA and RNA. Nucleotides are the monomeric units of nucleic acid polymers. Nucleotide polymers with less than about 100 monomeric units are termed oligonucleotides, while polynucleotides are larger. Such nucleotide polymers include deoxyribonucleic acid (DNA) and ribonucleic acid (RNA). Natural nucleotide polymers have a ribose-phosphate backbone. An artificial or synthetic nucleotide polymers is any nucleotide polymer that is polymerized in vitro and contains the same or similar bases but may contain a backbone of a type other than the natural ribose-phosphate backbone. These backbones include, but are not limited to: PNAs (peptide nucleic acids), phosphorothioates, phosphorodiamidates, morpholinos, and other variants of the phosphate backbone of natural nucleotide polymers.

Oligo- and polynucleotides may be delivered to express a particular encoded peptide or protein in a target cell. Where expression is desired the protein encoding nucleic acid may be incorporated into a vector or other expression vehicle, such as a virus. Further, such a vector or virus may be conjugated to a scaffold in an inventive conjugate.

Further, oligo- and polynucleotides may be delivered to suppress levels of a target molecule. For example, antisense oligonucleotides such as antisense RNA and small interfering RNA (siRNA) may be used in this context. Antisense technologies are known in the art as exemplified in references such as S. T. Crooke, (Ed.), Antisense Research and Application, Handbook of Experimental Pharmacology, Springer; 2002; S. T. Crooke, (Ed.), Antisense Drug Technology: Principles, Strategies, and Applications, Marcel Dekker, 2001; S. Agrawal (Ed.), Antisense Therapeutics, Methods in Molecular Medicine, Humana Press, 1996; and C. A. Stein and A. M. Krieg ads.), Applied Antisense Oligonucleotide Technology, Wiley-Liss, 1998.

Small interfering RNAs (siRNAs) became a part of antisense technology relatively recently and are particularly valuable as a therapeutic moiety due to their ability to induce sequence specific MRNA degradation. Further, siRNA modified with 2′-fluoro (2′-F) pyrinmidines have a half-life of over 24 hours in plasma with preserved functional activity (52) and are a useful therapeutic moiety. Further aspects of siRNAs are found in references such as N. Agrawal, et al., RNA Interference: Biology, Mechanism, and Applications, Microbiology and Molecular Biology Reviews, 2003, 67: 657-685; and T. Tuschl et al., Small Interfering RNAs: A Revolutionary Tool for the Analysis of Gene Function and Gene Therapy, Molecular Interventions, 2:158-167, 2002.

In one embodiment, small interfering RNAs are advantageously provided in the form of an siRNA expression cassette (SEC). Composition and generation of such cassettes is known in the art as exemplified in Miyagishi M. et al., Optimization of an siRNA-expression system with an improved hairpin and its significant suppressive effects in mammalian cells, J Gene Med., 2004, 6:715-23.

A therapeutic moiety may be a radionuclide illustratively including ¹⁹⁸Au, ¹⁹⁹Au, ¹¹¹Ag, ²¹²Bi, ⁶⁷Cu, ¹⁶⁵Dy, ¹⁶⁶Dy, ¹⁵⁹Gd, ⁶⁶Ho, ¹⁹²Ir, ¹⁴⁰La, ¹⁷⁷Lu, ¹⁰³Pd, ¹⁰⁹Pd, ¹⁴⁹pm, ¹⁸⁶Re, ¹⁸⁸Re, ¹⁰⁵Ru, ¹⁵³Sm, ⁹⁰Y, ¹⁶⁹Yb, and ¹⁷⁵Yb, and others known in the art, see for example, Ercan, M. T. et al., Therapeutic Radiopharmaceuticals, Current Pharm. Design, 6:1085-1121, 2000. A radionuclide therapeutic moiety is associated with an inventive conjugate as a chelated ion in one embodiment.

Further therapeutic moieties include pharmaceutical agents illustratively including anti-tumorals such as taxol, doxorubicin, vinca alkaloids, alkylating agents, methotrexate; anti-inflammatory agents, antivirals, antibiotics, antihistamines, and others such as listed in A. R. Gennaro, Remington: The Science and Practice of Pharmacy, Lippincott Williams & Wilkins, 20th ed. (2003); and J. G. Hardman et al., Goodman & Gilman's The Pharmacological Basis of Therapeutics, McGraw-Hill Professional, 10th ed. (2001).

Second Therapeutic Moiety

A second therapeutic agent may provide synergistic therapeutic benefits and may be any therapeutic agent as described above.

A second therapeutic moiety is optionally provided as a second therapeutic moiety conjugated to a scaffold.

In a further option, a second therapeutic moiety is provided in a pharmaceutical composition containing an inventive conjugate but not attached thereto.

Second agents included in treatment of cancer may be a chemotherapy pharmaceutical agent such as taxol, a radiotherapy agent such as a radionuclide, and an immunotherapy agent such as herceptin. In particular, a synergistic result may be obtained where an inventive conjugate sensitizes target cells to a second therapeutic agent or where a second therapeutic agent sensitizes target cells to a therapeutic moiety conjugated to an inventive conjugate.

Translocating Moiety

The term “translocating moiety” as used herein refers to a molecule, group of molecules, complex or substance conjugated to a scaffold moiety which enhances cellular penetration of a moiety to which it is conjugated into a cell.

One strategy to overcome poor intracellular delivery of a therapeutic moiety is to include a translocating species. Such a delivery strategy is based on properties of Protein Transduction Domains (PTDs), also called Translocating Peptides (TPs) or Cell Penetrating Peptides. PTDs are small protein domains, structurally different but having in common a high content of positively charged amino acids. PTDs have an ability to penetrate cellular and nuclear membranes, regardless of proliferation status of the cells. This unique ability of PTDs may be exploited to deliver proteins into cells both in vitro and in vivo. For example, a β-galactosidase, cloned in frame with a PTD from the HIV-Tat protein, was detected in 100% of blood cells and splenocytes 20 minutes post-intraperitoneal injection into C57BL/6 mice. (53) Some synthetic PTDs based on the structure of the Tat peptide demonstrated even higher efficiency in membrane penetration both in vitro and in vivo. (54) Further, PTDs have been used in delivering oligonucleotides successfully (55), down regulation of genes including Bcl-2 (56, 57) and P-glycoprotein (58) were successfully silenced. An advantage of some translocating moieties, including a Tat moiety is the ability of a Tat moiety appended to a bioactive molecule to enhance cell penetration without significant inhibition of the bioactivity of the bioactive molecule, see Schwartz, J. J. and Zhang, S., Peptide-mediated cellular delivery, Curr. Opin. Molecular Therapeutics, 2:162-167, 2000.

Examples of a translocating moiety include a cell penetrating peptide (CPP), a protein transduction domain (PTD), a Tat peptide, a Super-Tat peptide, MPG, VP22, antennapedia, and folic acid. Specific translocating moieties are illustrated in Schwarze, S. R. & Dowdy, S. F., In vivo protein transduction: intracellular delivery of biologically active proteins, compounds and DNA. Trends Pharmacol. Sci. 21, 45-48 (2000); Ho, A., et al., Synthetic protein transduction domains: enhanced transduction potential in vitro and in vivo. Cancer Res. 61, 474-477 (2001); Jarver, P. & Langel, U. The use of cell-penetrating peptides as a tool for gene regulation. Drug Discov. Today 9, 395-402 (2004); Lindsay, M. A., 2002, Peptide-mediated cell delivery: application in protein target validation, Curr. Opin. Pharmacol. 41:1023-1033; Wadia, J. S. et al., 2002, Protein transduction technology, Curr. Opin. Biotechnol., 13:52-56; and Schwartz, J. J. and Zhang, S., Peptide-mediated cellular delivery, Curr. Opin. Molecular Therapeutics 2:162-167, 2000.

A Tat peptide having the sequence GRKKRRQRRR (SEQ ID NO. 1) represents residues 48-58 of the HIV-1 Tat protein, has been demonstrated as a powerful nontoxic carrier for in vitro and in vivo delivery of protein, DNA, RNA and other macromolecules into all cells including across the blood-brain barrier (Schwarze 1999, 2000). This process is receptor and transporter independent and may target the lipid layer of the cell membrane directly. It has been successfully used to deliver DNA (Torchilin 2003, Eguchi A. 2001, Rudolph 2003) and antisense oligonucleotides (Zhang 2001).

In one embodiment a Tat peptide is a portion of a human immunodeficiency virus type 1 (HIV-1) Tat protein active to enhance uptake of a conjugate into a cell in vitro or in vivo. An exemplary Tat peptide which is a translocating moiety is GRKKRRQRRRP (SEQ ID NO. 2)—see Turner et al., Nucleic Acids Research, 33:27-42, 2005. Further Tat peptides which are translocating moieties included in an inventive conjugate are described in U.S, Pat. No. 6,316,003. In addition, modified Tat peptides may be used as a translocating moiety. For example, a Tat analog having the sequence YARAAARQARA (SEQ ID NO. 3) (Ho et al., 2001) is a translocating moiety which may be included in an inventive conjugate.

In one embodiment, a penetratin is a translocation moiety. Penetratins are translocating moieties described in detail in various references including Derossi et al., Trends Cell Biol., 1998, 8:84-87 and U.S. Pat. No. 6,821,948. An exemplary penetratin has the sequence RQIKIWFQNRRMKWKKGG (SEQ ID NO. 4)—see Turner et al., Nucleic Acids Research 33:27-42, 2005.

A further translocation moiety is a transportan. An exemplary transportan has the sequence GWTLNSAGYLLGKINLKALAALAKKIL (SEQ ID NO. 5)—see Turner et al., Nucleic Acids Research 33:27-42, 2005.

The term “endosome disruption moiety” as used herein refers to a molecule or group of molecules conjugated to a scaffold moiety which inhibits sequestration of an inventive conjugate in endosomes. For example, in one embodiment an endosome disruption moiety is the 10 amino acid Pol-loop segment of HSV-1 DNA polymerase —AVGAGATAEE (SEQ ID NO. 6)—see Hearn, A. R. et al., Journal of Biol. Chem. 279:51315-51322, 2004. Further exemplary endosome disruption moieties include peptides such as: KFTIVFPHNQKGNWKNVPSNYHYCP (SEQ ID NO. 7); EPVSLTLALLLGGLTMGGIAAGVGTGTTALVATQQAVGIGALFLGFLGAAGS TMGARS (SEQ ID NO. 8); AVGAIGALFLGFLGAAG (SEQ ID NO. 9); GLFEAIA EFIEGGWEGLIEGCA (SEQ ID NO. 10); GLFGAIAGFIENGWEGMIDGWYGFR (SEQ ID NO. 11); AVGIGALFLGFLGAAGSTMGAAS (SEQ ID NO. 12); FAGWIGLAALGVATAANVTAAVALVK (SEQ ID NO. 13); KVYTGVYPFMWG GAYCFCD (SEQ ID NO. 14); KLICTGISSIPPIRALFAAINIP (SEQ ID NO. 15); FFGAVIGTIALGVATATAAQIT (SEQ ID NO. 16); FAGVVIGLAALGVATATA AQVT (SEQ ID NO. 17); FIGAIIGGVALGVATATAAQIT (SEQ ID NO. 18); GLFGAIAGFIENGWEGMIDGWYGFRHQN (SEQ ID NO. 19); GLFGAIAGFIEN GWEGLVDGWYGFRHQN (SEQ ID NO. 20); GFFGAIAGFLEGGWEGMIAGWH GYTSHGB (SEQ ID NO. 21); FVAAIILGISALIAIITSFAVATTALVKEM (SEQ ID NO. 22); GLFGAIAGFIENGWEGMIDGGGC (SEQ ID NO. 23); GLFGAIAGFI ENGWEGMIDGWYG (SEQ ID NO. 24); GLFGAIAGFIENGWEGLAEALAEALE ALAAGGSC (SEQ ID NO. 25); EPVSLTLALLLGGLTMGGIAAGIGTGTTAL MATQQFQQLQAAVQDDLREVEKS (SEQ ID NO. 26); EPVSLTLALLLGGLTM GGLAAGVGTGTTALVATQQFQQLHAAVQDDLKEVEKS (SEQ ID NO. 27); EPVSLTLALLLGGLTMGGIAAGVGTGTTALVATQQFQQLQAAMHDDLKEV EKS (SEQ ID NO. 28); DYQCKVYTGVYPFMWGG AYCFCDSENT (SEQ ID NO. 29); DYTCKVFGGVYPFMWGGAQCFCDSENS (SEQ ID NO. 30); FAGVVLA GAALGVAAAAQI (SEQ ID NO. 31); FAGVVLAGAALGVATAAQI (SEQ ID NO. 32); GLFGAIAGFIENGWEGMIDG (SEQ ID NO. 33); GIFGAIAGFIENGW EGMIDG (SEQ ID NO. 34); GLFGAIAGFIEGGWTGMIDG (SEQ ID NO. 35); GLFGAIAGFIEGGWEGMVDG (SEQ ID NO. 36); GLFGAIAGFIEGGWEG LVDG (SEQ ID NO. 37); and GFFGAIAGFLEGGWEGMIAG (SEQ ID NO. 38). These and other such endosome disruption moieties are further described in references including: R. Schlegel et al., J. Biol. Chem. 259, 4691-94, 1984; Plank et al., J. Biol. Chem. 269:12918-12924, 1994; E. Mastrobattista et al., J. Biol. Chem. 277:27135-27143, 2002; J. M. White, Science 258, 917-24, 1992; J. White, et al., Q. Rev. Biophys. 16, 151-95, 1983; S. M. W. van Rossenberg et al., J. Biol. Chem. 277:45803-45810; P. L. Yeagle et al., Biochim. Biophys. Acta 1065, 49-53, 1991; P. Midoux et al., Nucl. Acids Res. 21, 871-78, 1993; E. Wagner et al., Proc. Nat. Acad. Sci. USA 89, 7934-38, 1992; S. Soukchareun et al., Bioconj. Chem. 6, 43-53, 1995; J. L. Nieva et al., Biochem. 33, 3201-09, 1994; J. D. Lear et al., J. Biol. Chem. 262, 6500-05, 1987; and J. S. Jones et al., J. Virol. 67, 67-74, 1993; each of which is incorporated herein by reference. Non-proteinaceous endosome disruptors operative herein illustratively include ethylenimine polymers (PEIs); lipopolyamines such as alkylated amidoglycyl spermines, alkylamines, alkylamides, and phosphatidyl ethanolamines.

Reporter Moiety

In addition an inventive conjugate may include a reporter moiety. A reporter moiety aids in monitoring delivery of an inventive conjugate to a target. In one embodiment a reporter moiety allows non-invasive monitoring of an inventive conjugate. Noninvasive in vito imaging includes techniques such as gamma camera imaging of gamma ray emissions, single-photon emission computed tomography (SPECT), and positron emission tomography (PET) of a radiolabeled compound, transmission computed tomography (CT), optical imaging such as imaging of bioluminescent and fluorescent compounds, magnetic resonance imaging (MRI) and magnetic resonance spectroscopy (MRS). A reporter moiety is a spectroscopically active moiety such as a radioactive moiety for the PET and SPECT imaging, non-radioactive moiety for MI and MRS contrast imaging, or a fluorescent reporter for optical imaging.

In one embodiment of an inventive conjugate a reporter is a metal M having the formula M²⁺or M³⁺. In one embodiment, M is a lanthanide, a transition metal or a heavy metal, such as Gd3+for MRI contrast imaging. Typical monitoring/imaging radionuclides include but are not limited to ^{99m}Tc, ⁹⁶Tc, ⁹⁵Tc, ¹¹¹In, ⁶²Cu, ⁶⁴Cu, ⁶⁷Ga, ⁶⁸Ga, ²⁰¹Tl, and ¹⁹²Ir.

Particular fluorescent reporters include compounds such as fluorescein isothiocyanate (FITC), rhodamine, TAMR and other such fluorescent labels such as described in The Handbook—A Guide to Fluorescent Probes and Labeling Technologies, 10^{th} Ed., Invitrogen Corp., Carlsbad, Cailf., for optical imaging of intracellular delivery of an inventive conjugate.

In a further embodiment, a chemiluminescent reporter such as luminol may be used.,

A particular reporter scheme according to the present invention delivers a reporter moiety as detailed herein, in conjunction with a cargo species that binds to an intracellular component associated with a particular cell type or metabolic state. Upon cargo species binding, the reporter moiety is preferentially within the cell at the time of imaging as compared to a cell lacking the intracellular component. By way of example, cancer cells are known to over-express survivin, an inventive conjugate including an antisense oligonucleotide or interacting peptide for survivin is conjugated to a scaffolding specie coordinating a Gd³⁺ ion. It is appreciated that any intracellular molecule over expressed or under expressed in a target cell is exploited for cellular imaging. Such molecular targets in cancer in addition to survivin illustratively include ras, hTERT and phospho-Akt. Cargo species suitable for binding such intracellular targets illustratively include small interfering RNAs, anti-sense oligonucleotides, peptides capable of recognizing and interacting with the target molecule, antibodies, and antibody fragments. Subsequent MRI imaging shows greater contrast for cancerous cells as compared to surrounding normal cells.

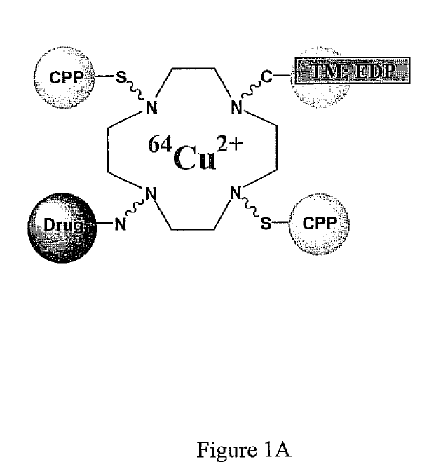

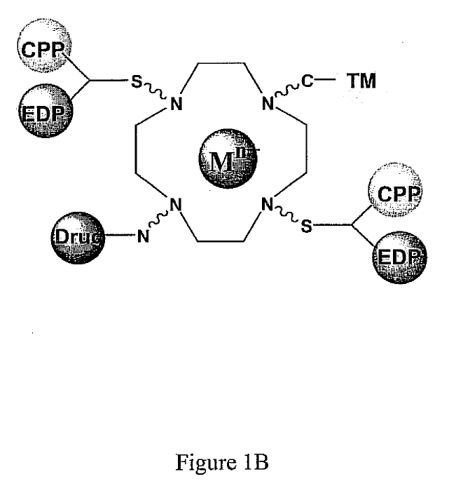

FIG. 1A is a schematic illustration of an exemplary inventive conjugate in which a cyclen scaffold moiety is conjugated to two translocating moieties or cell penetrating peptides (CPP), a targeting moiety or targeting molecule (TM) and a therapeutic moiety or drug. Also shown is an endosome disrupting moiety or endosome disrupting peptide (EDP). Optionally, one cargo moiety is directly bonded to a second cargo moiety, either directly or through a linker. As depicted here, an endosome disrupting moiety is bonded to a targeting moiety. Further illustrated is a reporter moiety, ⁶⁴Cu, associated with the conjugate. Linkage between the cargo moieties and the scaffold moiety is shown schematically in this illustration.

In a particular example, an inventive conjugate includes a protein transfection domain (PTD) of the HIV-Tat protein which is a translocation moiety and a macrocyclic scaffold including a reactive linker moiety, for example CYCLEN. In such a conjugate a macrocyclic core serves as a scaffold for assembly of macromolecules with different functional properties. In particular, a translocating peptide (Tat) serves as an engine for intracellular delivery of a “cargo” such as DNA, RNA or peptide. In addition, a cellular or organelle targeting moiety is optionally attached to the scaffold moiety, for instance a peptide fragment of a receptor ligand, an aptamer or the like. In addition, an endosome disruption moiety may be incorporated into the conjugate. Further, the composition may serve as a chelator to bind a radiometal for molecular imaging, in a particular example ⁶⁴Cu for microPET imaging and ¹¹¹In for microSPECT/CT scanning. Advantages of this approach include high stability of the conjugate; relative ease of assembly; possibility of monitoring delivery in vivo using noninvasive imaging techniques; and potential to target conjugate to particular cells, such as cancer cells.

FIG. 1B is a schematic illustration of an inventive conjugate. In such an embodiment, multiple cargo moieties are bonded to a scaffold moiety through an atom which is a macrocycle ring member. Linkage to the nitrogen atoms in the macrocycle scaffold is shown schematically. Two translocating moieties (CPP) and two endosome disruption moieties (EDP) are shown attached to the scaffold in an opposing orientation. Further illustrated are a targeting moiety (TM) and a therapeutic moiety (drug) attached to the scaffold through linkers.

Pharmaceutical Compositions

An inventive pharmaceutical composition includes an inventive conjugate and a pharmaceutically acceptable carrier.

The term “pharmaceutically acceptable carrier” as used herein is intended to refer to a carrier or diluent that is generally non-toxic to an intended recipient and which does not significantly inhibit activity of an active agent included in the composition.

An inventive composition is suitable for administration to patients by a variety of routes local and systemic, illustratively including intravenous, oral, parenteral, transdermal, intrathecal, intraventricular, ocular, otic, nasal, anal, genital, buccal, and mucosal. Inventive compositions may also be administered as inhalants. Further, an inventive composition may be administered acutely or chronically.

In one embodiment, an inventive composition is administered to a tumor, for instance by intratumoral, intravenous, intraperitoneal or intrathecal injection.

Compositions suitable for delivery may be formulated in various forms illustratively including physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers; diluents; solvents; or vehicles include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of the required particle size in the case of dispersions, and by the use of surfactants such as sodium lauryl sulfate.

Compositions suitable for injection optionally include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles for injectable compositions include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged delivery of an injectable pharmaceutical form can be brought about by the use of agents delaying absorption, for example, aluminum monostearate and gelatin. Solid dosage forms for oral administration include capsules, tablets, pills, powders, and granules. In such solid dosage forms, an inventive conjugate is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymefthylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, plant starches, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, and glycols (h) adsorbents, as for example, kaolin and bentonite, and (i) lubricants, as for example, talc, calcium stearate, magnesium stearate, solid polyethylene glycols, sodium lauryl sulfate, or mixtures thereof In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied with to a thickness such that the entire coating does not dissolve in the gastrointestinal fluids at pH below 35 associated with stomach acids, yet dissolves above pH 5 in the small intestine environment. It is expected that any anionic polymer exhibiting a pH-dependent solubility profile is readily used as an enteric coating in the practice of the present invention to achieve delivery of the active to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phtialate, cellulose acetate trimellitate, hydroxypropylmethyl cellulose phthalate, hydroxypropylmethyl cellulose succinate and carboxyrnethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particularly preferred enteric coating material for use herein is those acrylic acid polymers and copolymers available under the trade name EUDRAGIT®, Roehm Pharma (Germany). The EUDRAGIT® series L, L-30D and S copolymers are most preferred since these are insoluble in stomach and dissolve in the intestine.

The enteric coating provides for controlled release of the active agent, such that release is accomplished at a predictable location in the lower intestinal tract below the point at which drug release would occur absent the enteric coating. The enteric coating also prevents exposure of the active agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated solid dosages of the present invention allow optimization of drug absorption, active agent protection, and safety. Multiple enteric coatings targeted to release the active agent at various regions in the lower gastrointestinal tract would enable even more effective and sustained improved delivery throughout the lower gastrointestinal tract The enteric coating optionally contains a plasticizer to prevent the formation of pores and cracks that allow the penetration of the gastric fluids into the solid dosage. Suitable plasticizers illustratively include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The enteric coating is applied to a solid dosage using conventional coating methods and equipment. For example, an enteric coating can be applied to a solid dosage using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in L. V. Allen et al., Ansel's Pharmaceutical Dosage Forms and Drug Delivery Systems, Lippincott Williams & Wilkins, 8th ed., Philadelphia, (2004).

Liquid dosage forms for oral administration include a pharmaceutically acceptable carrier formulated as an emulsion, solution, suspension, syrup, or elixir. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dirnethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfiming agents.

Suspensions, in addition to an inventive conjugate, may contain suspending agents, as for example, ethoxylated isostearyl alcohols, polyoxyethylene sorbitol and sorbitolan esters, microcrystalline cellulose, aluminum metahydroxide, bentonite, agar-agar and tragacanth, or mixtures of these substances, and the like.

Further examples and details of pharmacological formulations and ingredients are found in standard references such as: A R. Gennaro, Remington: The Science and Practice of Pharmacy, Lippincott Williams & Wilkins, 20th ed, (2003); L. V. Allen et al., Ansel's Pharmaceutical Dosage Forms and Drug Delivery Systems, Lippincott Williams & Wilkins, 8th ed., Philadelphia (2004); J. G. Hardman et al., Goodman & Gilman's The Pharmacological Basis of Therapeutics, McGraw-Hill Professional, 10th ed. (2001).

Methods

In one embodiment, inventive compositions are included in a method of treating a pathological condition of a patient. Such a method includes administering a therapeutically effective amount of an inventive composition to a patient in need of such treatment.

An effective amount of an inventive conjugate is an amount that when administered to a patient or subject ameliorates a condition or symptom of the patient.

A patient or subject to be treated includes humans, but also includes other species such as non-human primates, dogs, cats, cattle, pigs, horses and rodents.

It is appreciated that an inventive conjugate is also useful in vitro, for instance as a research tool for delivery of a cargo moiety to a cell.

Embodiments of inventive compositions and methods are illustrated in the following examples. These examples are provided for illustrative purposes and are not considered limitations on the scope of inventive compositions and methods.

### EXAMPLES

### Example 1

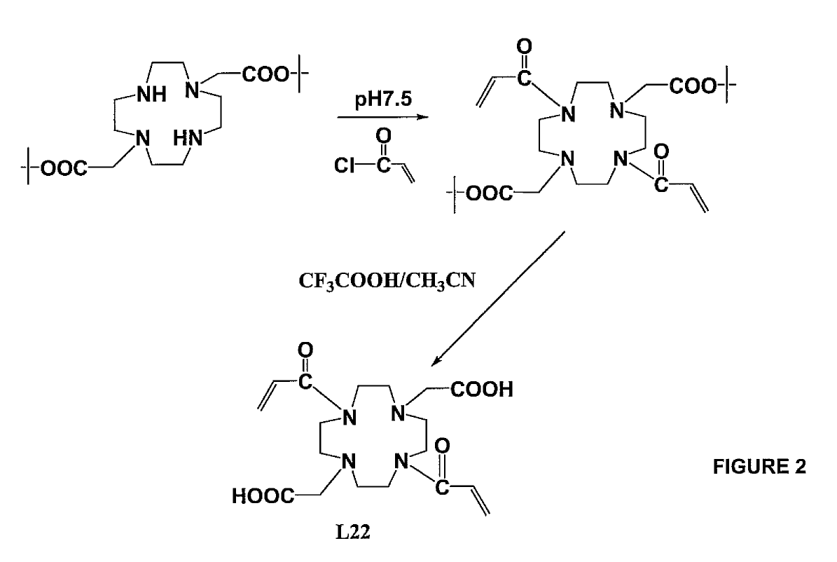

FIG. 2 shows an example of a cyclen scaffold moiety having two N-linked—CH2—COO- moieties: 1,4,7,10-tetraazacyclododecane-1,7-bis(t-butyl acetate), which may be synthesized from a commercial available precursor, such as CYCLEN from Macrocyclics, Dallas, Tex. This compound may be modified by conjugation of two further linker moieties, —CO—CH—CH2, as shown in the figure.

### Example 2

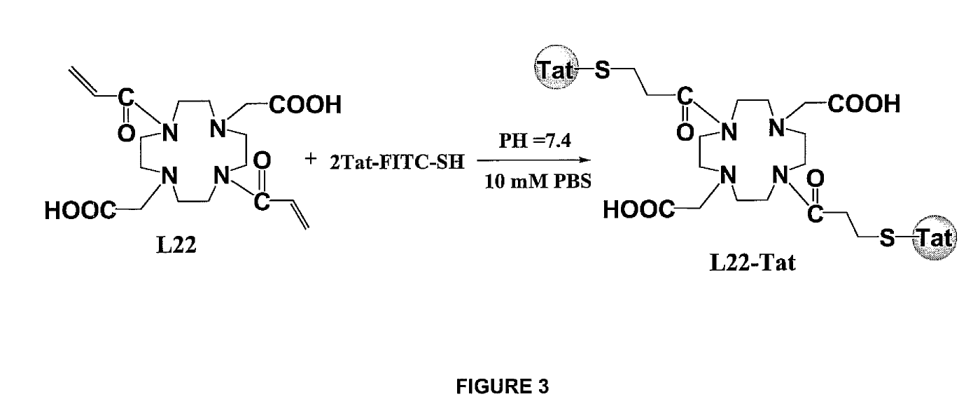

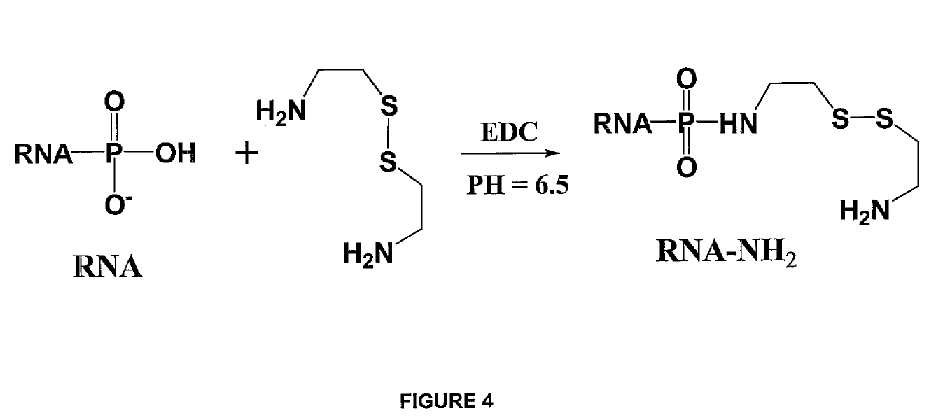

FIG. 3 shows an example of a reaction for conjugation of a translocation moiety to a scaffold. In the particular example shown, two translocation moieties—Tat -having pendant reporter moieties—FITC—are conjugated to the scaffold.

### Example 3

FIG. 3 shows an exemplary modification of an oligonucleotide to include a linker. Here, the linker is reacted in the presence of a water-soluble carbodiimide coupler, 1-ethyl-3-(3-dimethylaminopropyl)-carbodiimide (EDC) at pH 6.5 to form an amide linkage between the oligonucleotide and the linker. The resulting modified oligonucleotide includes the linker having a reactive functional group in the form of an amino group.

### Example 4

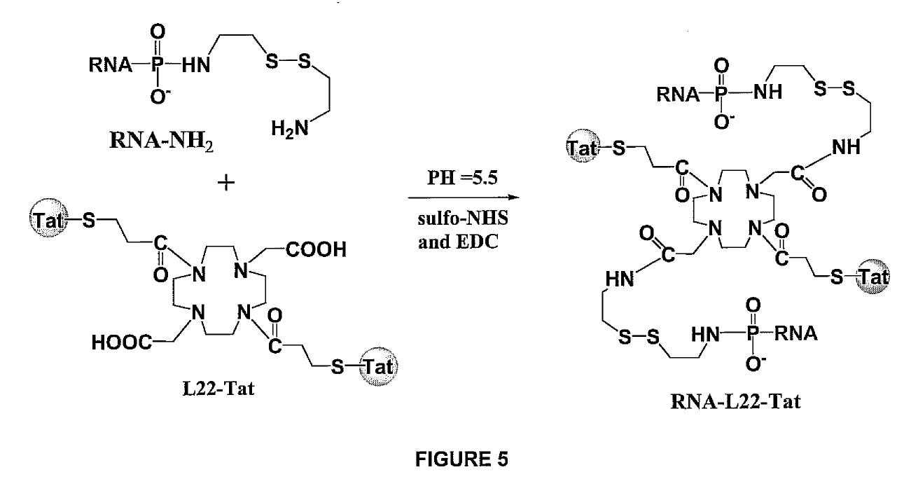

An oligonucleotide modified as described in Example 3 is illustratively attached to a scaffold moiety as shown in FIG. 5. In this example, an oligonucleotide-linker complex having a free amino group on the linker is reacted with a scaffold moiety having a free carboxyl group in the presence of a carbodiimide coupler and N-hydroxysuccinimide (NHS) at 5.5.

### Example 5

A cyclen scaffold is modified to include one to four N-linked —CH2COOH groups, yielding the compound 1,4,7,1 0-teaazacyclododecane-N,N′,N″,N′″-tetraacetic acid (DOTA). Synthetic methods for DOTA are known in the art, as exemplified in U.S. Pat. No. 5,428,156. Briefly, as described therein, a cyclen macrocycle is cyanomethylated in the presence of acid, such as sulfuric acid, formaldehyde and a cyanide, such as sodium or potassium cyanide. The resulting compound is hydrolyzed using a base, such as LiOH and ten acidified using an acid, such as HCl. The resulting compound is dried, resuspended in an alcohol, such as methanol and precipitated, for instance with acetone. A similar method is used to synthesize an analogous cyclam derivative, 1,4,8,11-tetraazacyclododecane-1,4,8,1 1-tetraacetic acid as described in the same reference.

### Example 6

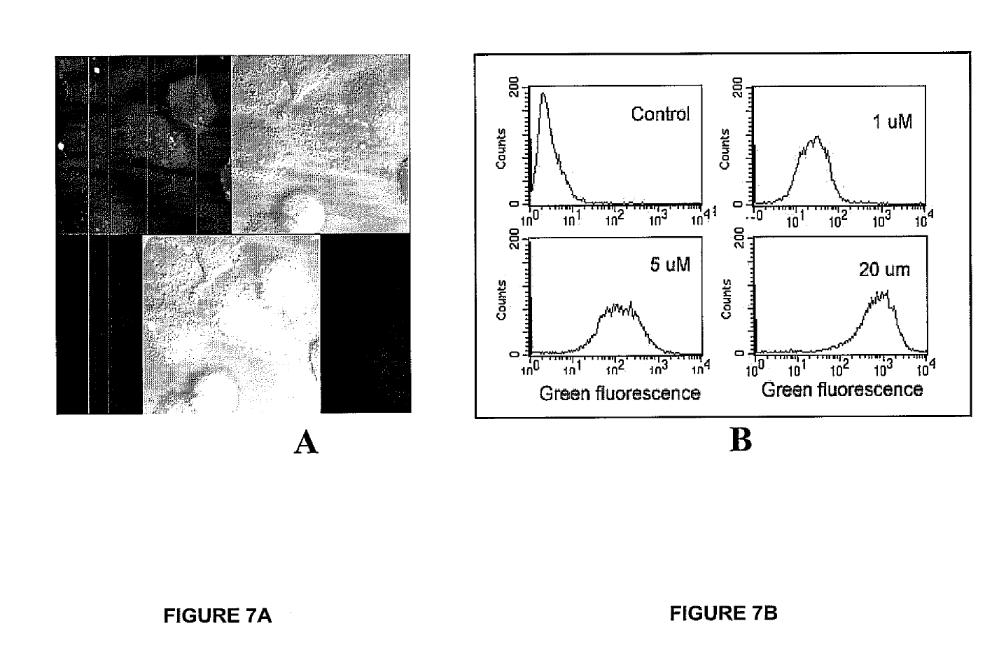

A conjugate SK-BR-3 and MCF-7 breast cancer cells are treated with increasing concentrations of FITC labeled Tat-CYCLEN (TC) conjugates for 20 hours. SK-BR-3 cells are incubated with increasing concentrations of FITC labeled TC (TC-FITC) conjugate for 2 hours and examined by confocal microscopy (FIG. 7A) and flow cytometry (shown in FIG. 7B). FIG. 7B flow cytometry data demonstrates that incubation of cells with TC-FITC produces a dose-dependent increase in the percentage of FITC positive cells as well as median fluorescence. To confirm intracellular localization of the fluorescence, confocal microscopy of living cells may be used. This technique allows performance of a full thickness scan of the cells to differentiate between surface and intracellular fluorescence. SK-BR-3 cells, grown on a glass cover slip, are incubated with 1 micromolar TC-FITC. Confocal microscopy shows preferential nuclear accumulation of the fluorescence.

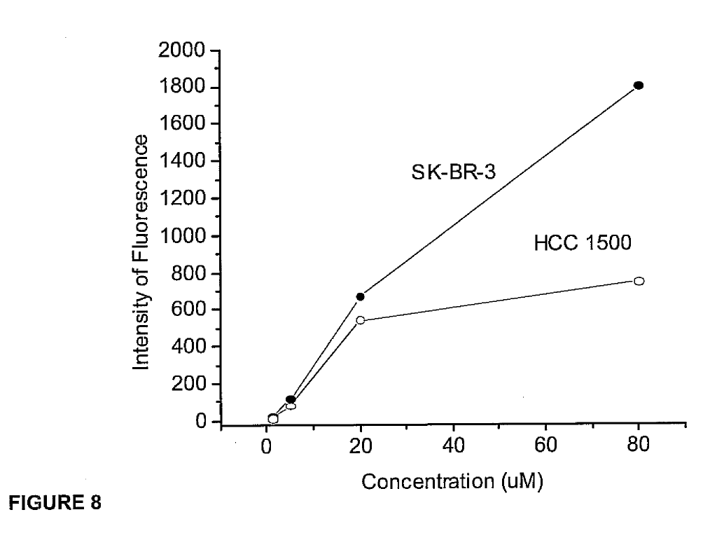

FIG. 8 shows quantitation of uptake of FITC-labeled TC conjugates into SK-BR-3 and HCC1500 cells.

### Example 7

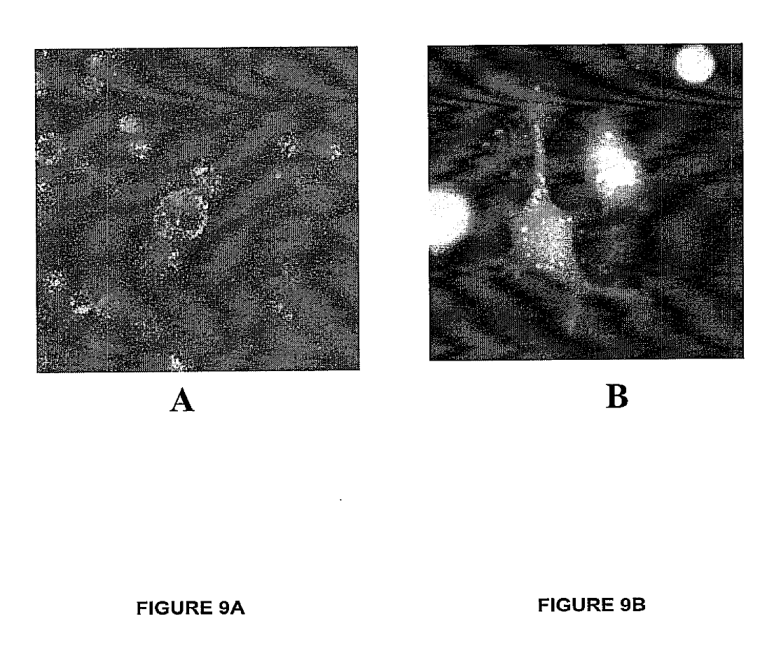

Unmodified antisense oligonucleotides directed to survivin, a tumor antigen, are conjugated to a fluorescently labeled Tat-CYCLEN (TC-1-TAMR). Conjugates are added to SK-BR-3 cells and grown on glass slides for 48 hours in the presence of 300 nanomolar concentration of the conjugate. Survivin-antisense oligonucleotide-TC conjugates are added to serum containing complete growth medium in a volume of 5 microliters for a final concentration of 300 nanomolar. Confocal microscopy is performed at 1 and 3 hours following addition of the conjugate to the medium. Intracellular fluorescence is seen in 100% of the cells after 1 hour. Initial non-uniform distribution of fluorescence is observable and may represent accumulation of the conjugates in endosomes in addition to lower intensity random distribution of the fluorescence throughout the cell as shown in FIG. 9A. At 3 hours intensity of fluorescence increases and becomes more uniformly distributed through the cell as shown in FIG. 9B.

### Example 8

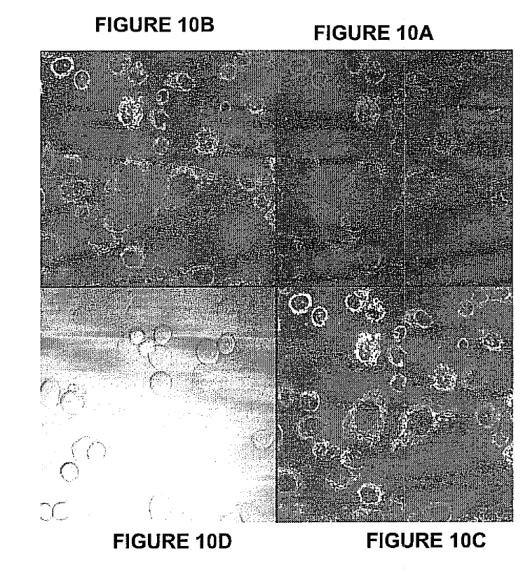

Survivin antisense oligonucleotide-TC-TAMR conjugate is added to SK-BR-3 cells grown on cover slips at a concentration of 300 nanomolar. Four hours after addition of the conjugate, confocal microscopy is performed to assess localization of the conjugate. FIG. 10A shows localization of the conjugate within cells. FIG. 10B shows localization of FM1-43, an endosomal marker. FIG. 10C shows overlapping conjugate and FM1-43 signals. FIG. 10D shows a light micrograph of the cells in panels 10A-10C.

### Example 9

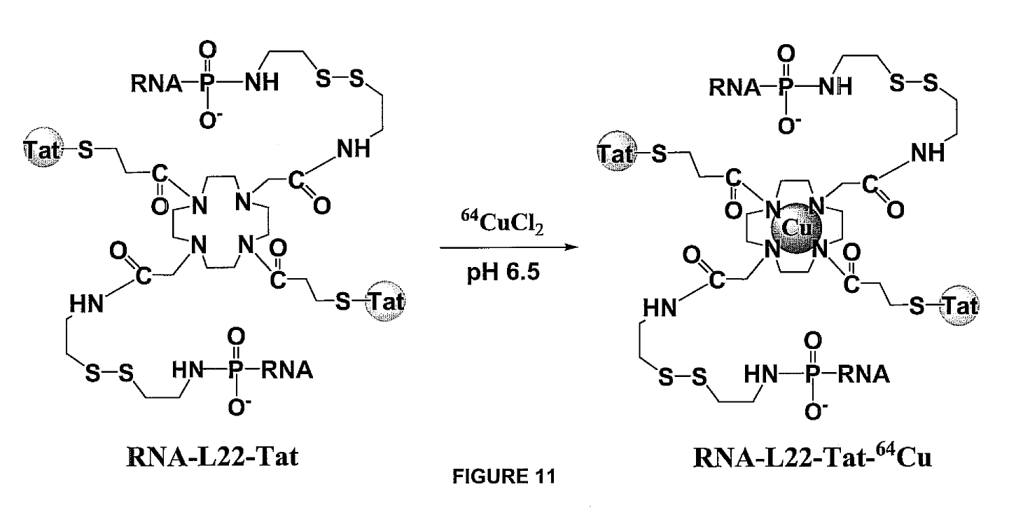

An inventive conjugate including a tetrazamacrocyclic scaffold conjugated to two Tat peptides and two oligoribonucleotides is shown in FIG. 11 (RNA-L22-Tat). A radioactive metal is incorporated in the conjugate by incubation with ⁶⁴CuCl₂ at pH 6.5 to produce RNA-L22-Tat-⁶⁴Cu.

### Example 10

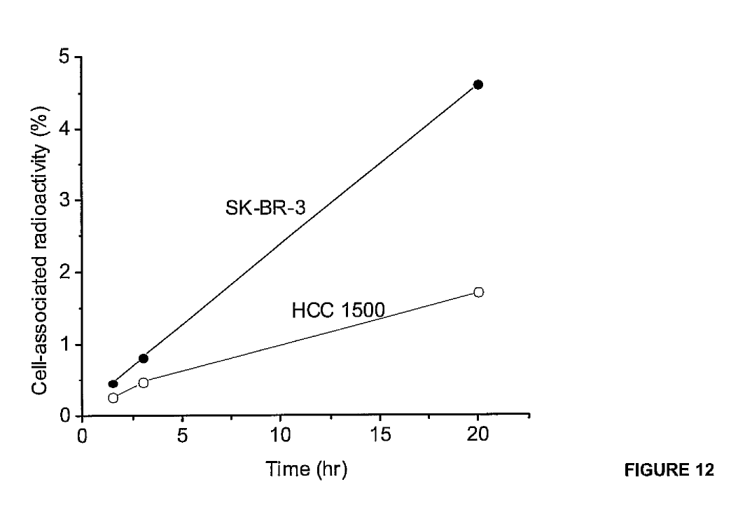

Uptake of a radiolabeled inventive conjugate into cultured SK-BR-3 and HCC-1500 cells is measured at various times of incubation. Cell-associated radioactivity increases with time as shown in FIG. 12.

### Example 11

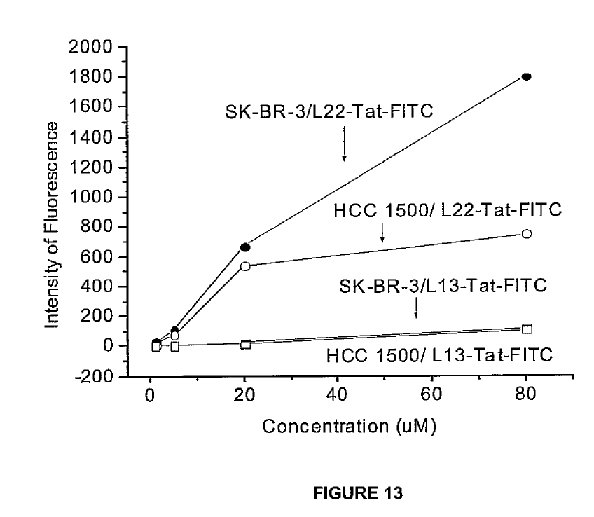

FITC-labeled TC conjugates containing 1 Tat peptide (L13-Tat-FITC) or 2 Tat peptides (L22-Tat-FITC) are synthesized and incubated with SK-BR-3 or HCC-1500 cells at various concentrations. Uptake of the conjugates into cells is measured as intensity of fluorescence. FIG. 13 shows increasing fluorescence intensity with increasing concentration as well as effects of more than one Tat peptide on uptake.

### Example 12

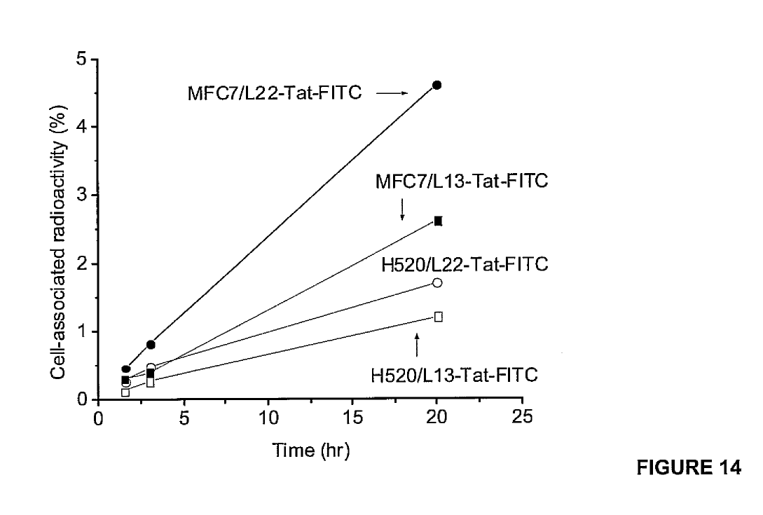

FITC-labeled TC conjugates containing 1 Tat peptide (L13-Tat-FITC) or 2 Tat peptides (L22-Tat-FITC) are synthesized and labeled with 64Cu. The conjugates are incubated with MFC7 or H520 cells for various times, Uptake of the conjugates into cells is measured as cell-associated intensity. FIG. 14 shows increasing cell-associated radioactivity with increasing time as well as effects of more than one Tat peptide on uptake.

### Examnple 13

A conjugate including TC, a fluorescent marker TAMRR, and a commercial 25 base pair oligonucleotide aptamer having a strong affinity for cancer cells (Aptamera Inc., KY) is synthesized.

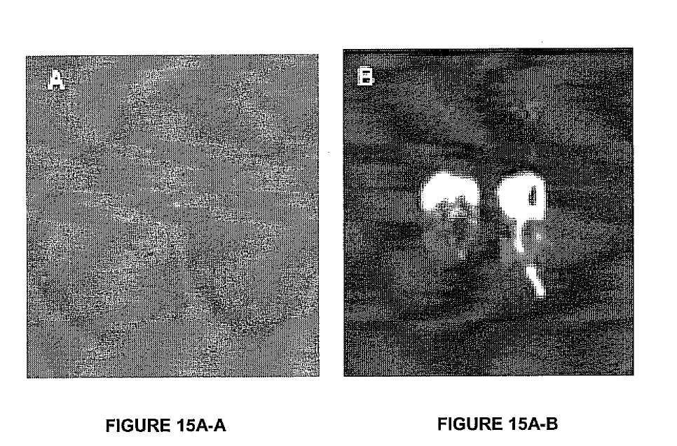

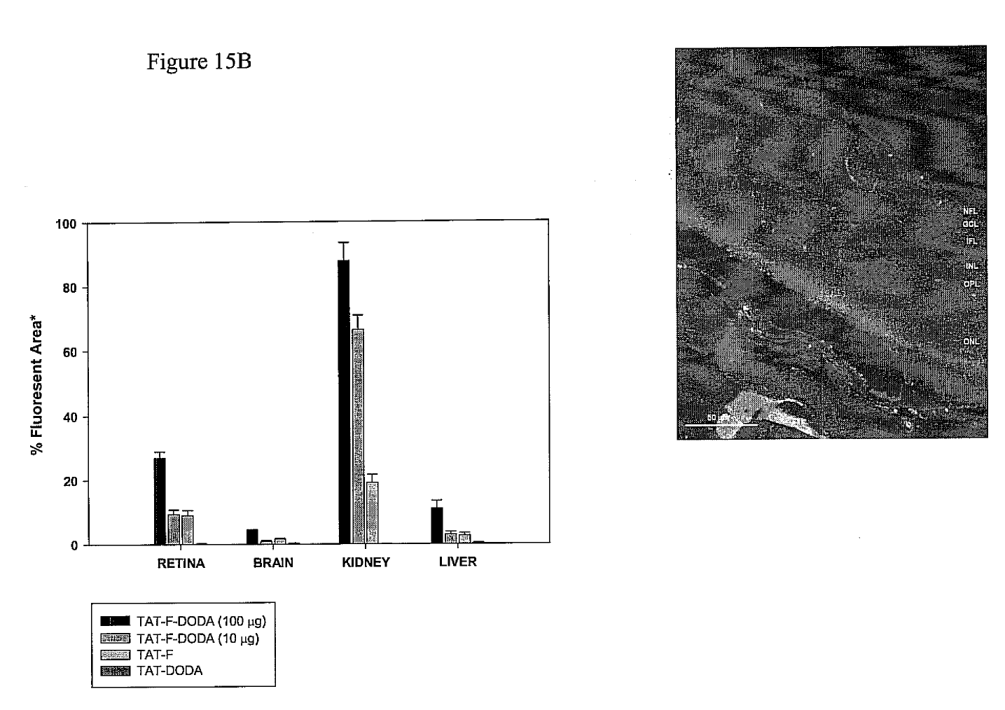

The TC-TAMRR-aptamer conjugate is delivered intracellularly in vitro. Cells are grown on a glass cover slip for 48 hours. TR labeled TC conjugates are added to the FBS-containing growth medium in the amount corresponding to 700 nanograms of oligonucleotide per milliliter. Uptake of conjugates is observed after 1.5 hours using confocal microscopy as shown in FIG. 15A. Similar results are obtained after 24 hours of incubation.

### Example 14

Experiments are performed to determine the uptake of a TC-aptamer conjugate in vivo. A TC aptamer conjugate is radiolabeled with Cu-64 and injected intravenously in mice with spinal cord metastases from breast cancer as well as a control mouse. MicroPET scans are performed 24 hours later and images confirm preferential accumulation of radiolabeled TC-aptamer conjugates in the tumor of the tumor-containing mouse. Images obtained from such studies are exemplified in FIG. 15B and show a strong preferential accumulation of conjugates in the tumors compared to other tissues in the mice.

### Example 15

An inventive conjugate is constructed including a macrocyclic core CYCLEN, a translocation moiety HIV-Tat, and an antisense oligonucleotide directed to survivin. Different types of modified antisense oligonucleotides may be employed, including phosphorothioate and morpholino modified antisense oligonucleotides (Gene Tools).

Advantages of phosphorothioate antisense oligonucleotides are that they have been extensively studied in preclinical models and in clinical trials and have no significant toxicity at particular dosage ranges. Further, they are more stable than unmodified antisense oligonucleotides.

A particular survivin antisense oligonucleotide used in an inventive conjugate is 5′-ATCGTGTGCTATTCTGTGAATT-3′(SEQ ID NO. 39). This sequence is identical to one described by Li et al. (32) except for a 4 base spacer at the 5′end which is used for attachment to the TC compound. In this example a negative control oligonucleotide is used having the sequence 5′-ATCGTAAGCTGTTCTATGTGTT-3′ (SEQ ID NO. 40).

In further examples morpholino antisense oligonucleotides are used. These modified oligonucleotides are extremely stable and potent, achieving efficient gene suppression for well over one week after a single administration, Further, they are uncharged, minimizing their interaction with proteins and other non-target moieties, In this example the morpholino modified antisense oligonucleotide studied is 5′-CCCATGCCGCCGCCGCCACCTCTG-3′(SEQ ID NO. 41). This survivin sequence is in position −19 through +6 of Genbank file NM001168.

The stability of siRNA in human plasma is improved by introduction of 2-fluoro (2′F) pyrimiidines into the sense and antisense strands. In the present example, two separate 21 nucleotide strands are used. These sequences described by Ling et al. (48) have the sequence sense 5′-GCGCCUGCACCCCGGAGCGdTdT-3′ (SEQ ID NO. 42) and antisense 5′-CGCUCCGGGGUGCAGGCGCdTdT-3′(SEQ ID NO. 43), scrambled control sense 5′-CAGUCGCGUUUGCGACUGGdTdT-3′ (SEQ ID NO. 44), scrambled control antisense 5′-CCAGUCGCAAACGCGACUGdTdT-3′ (SEQ ID NO. 45).

### Example 16

Inventive constructs are incubated with various cell lines in vitro to examine their uptake. Cell lines used include those easily transfected such as AGK293, intermediate difficulty of transfection MCF-7, SK-BR-3 and MCF-10A, and difficult to transfect cells including HCC1500 and primary human skin fibroblast cell lines. Cells are incubated with increasing concentrations of FITC labeled TC-oligonucleotide constructs or TC-siRNA constructs for 2 hours. Cells are analyzed by flow cytometry and confocal microscopy to determine uptake and localization of the constructs.

### Example 17

Efficiency of inventive conjugates in gene suppression are examined using conjugates including antisense oligonucleotides directed to survivin and siRNA conjugates directed to survivin as described above. MCF-HER29 cells are used since these cells overexpress ERBB2 in the presence of doxycycline. These cells upregulate survivin protein approximately 20 times in the presence of doxycycline. High expression of survivin in these cells allows examination of various conjugates over a wide range of concentrations. Doxycycline treated MCF-ATR29 cells are incubated in the presence of amounts of conjugates adjusted to provide final antisense oligonucleotides or siRNA concentrations of 10, 30, 50, 100, 300 and 500 nanomolar. Conjugates containing control oligonucleotides and siRNA are also examined. Whole cell protein extracts are collected every 24 hours after transduction for a total of 7 days to determine the time, course and duration of survivin suppression. The extracts are analyzed for survivin expression by Western blotting according to standard techniques. Further cell lines which express survivin breast cancer cell lines SK-BR-3, MDA-NB-231, HCC1500, lung cancer cell lines NAH-520, NIH-460, bladder cancer cell lines UMUC-3 are also used in assays described above.

### Example 18

In Vivo Delivery

A xenograph model of cancer is used to examine the efficacy of inventive constructs in suppressing survivin expression. Six week old female nude mice are obtained from Harlan Laboratories one week prior to xenograph injection of MCFHER29 breast cancer cells. All mice are primed with 17 β-estradiol (Innovative Research of America) applied subcutaneously in a biodegradable sustained-release carrier binder (1.7 milligrams of estradiol per pellet) to promote tumor growth. Mice are inoculated s.q. with 1×10⁷ MCF-AGR 29 cells. For ERBB2 induction animals are given doxycycline in drinking water (2 milligrams/milliliter in 5% sucrose). Tumor measurements are performed twice weekly and conjugates are administered when tumors reach the size of 100 mm³ which takes approximately 2 weeks.

### Example 19

An animal model of cancer as described in the above example is used to confirm delivery of TC-oligonucleotides and TC-siRNA to the tumors in the animals and to determine the extent of survivin suppression. The various TC-oligonucleotides and TC-siRNA conjugates and the corresponding controls are labeled with a fluorescent marker. In this example, the fluorescent marker is attached to the oligonucleotide moiety. Conjugates may be additionally labeled with long half-life radionuclide ⁶⁴Cu or ¹¹¹In for direct molecular imaging.

Animals receive injections of the conjugates intravenously in doses ranging from 200 micrograms per kilogram to 20 milligrams per kilogram, and control animals receive phosphate buffered saline. Antisense oligonucleotide constructs are injected in amounts starting at 200 micrograms per kilogram and titrated down in 5x increments. siRNA will start at 200 micrograms per kilogram and is titrated down in 5x increments, Two animals per conjugate type are included at each dosage level. In vivo biodistribution of the radiolabeled TC conjugates is assessed at 24, 48 and 72 hours after injection using a microPET instrument and a microSPECT/CT scanner. Tumors are extracted and evaluated for intracellular fluorescence distribution using fluorescent microscopy and for survivin expression using western blotting. Other organs are also collected in order to assess conjugate concentrations and penetrations into these tissues.

### Example 20

Animals prepared as described above are injected with a single intravenous injection of TC-survivin antisense oligonucleotide and siRNA conjugates. Two anirnals from each treatment group are sacrificed on days 1, 3, 5 and 7. Protein extracts are prepared from tumor tissues and analyzed for survivin expression using Western blotting.

### Example 21

Optionally a second therapeutic agent is used with inventive conjugates. MCF-7 and SK-BR-3 breast cancer cells and the “normal” breast cancer epithelial cell line MCF-10A are treated with increasing taxol concentrations in the presence or absence of various concentrations of anti-survivin TC conjugates described herein. Cells are harvested 48 to 72 hours after taxol treatment and cell survival/proliferation is tested using a standard MTT assay. Induction of apoptosis is evaluated using an annexin V staining, caspase 3/7 activation or TUNEL assay.

### Example 22

Inventive conjugates are used in a rheumatoid arthritis model-K/BxN murine model. Rheumatoid arhritis is an autoimmune disease with an intra-articular inflammation and synovial hyperplasia that results in progressive degradation of cartilage and bone. There are several rheumatoid arthritis animal models available. In a K/BxN murine model of rheumatoid arthritis, an auto antibody against glucose-6-phosphate isomerase (GPI) causes joint specific inflammation and destruction.

An anti-GPI antibody is conjugated to 1,4,7,10-tetraazacyclododecane-N,N′,N″,N′″-tetraacetic acid (DOTA). The resulting conjugate is labeled with ⁶⁴Cu, a cyclotron produced positron emitting isotope with a half-life of 12.7 hours.

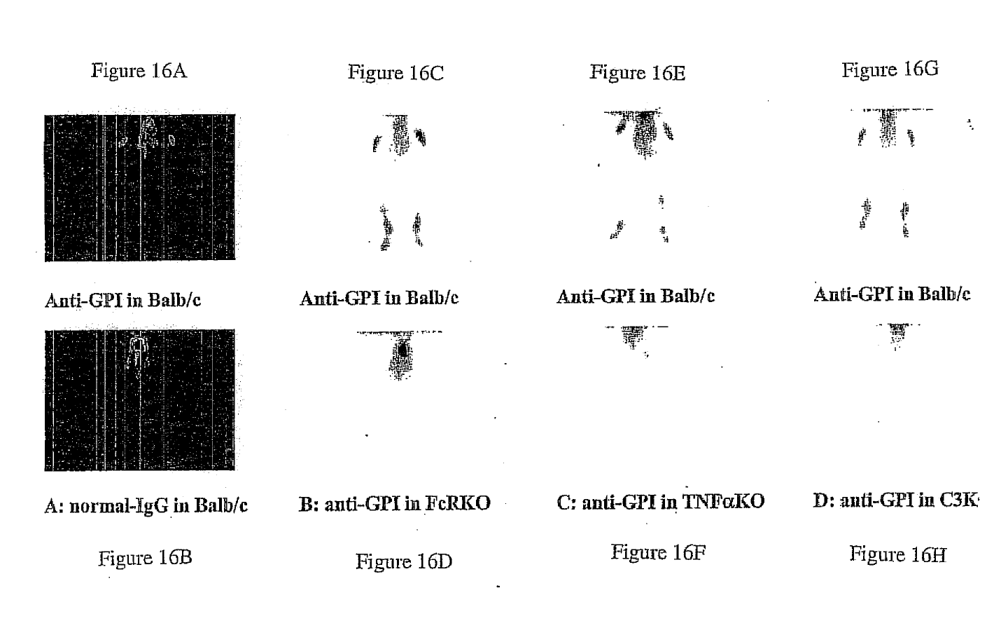

FIGS. 16A, C, E, and G shows that GPI-specific autoantibodies localize to distal joints of normal mice within minutes and induce rapid joint inflammation with onset by 24 hours (Wipke, 2002). FIGS. 16D, F and H show much lower levels of anti-GPI in distal joints of three types of knockout mice, including FcR knockout, TNF-α knockout and C3 knockout mice respectively. FIG. 16B shows distribution of normal IgG in a control mouse.

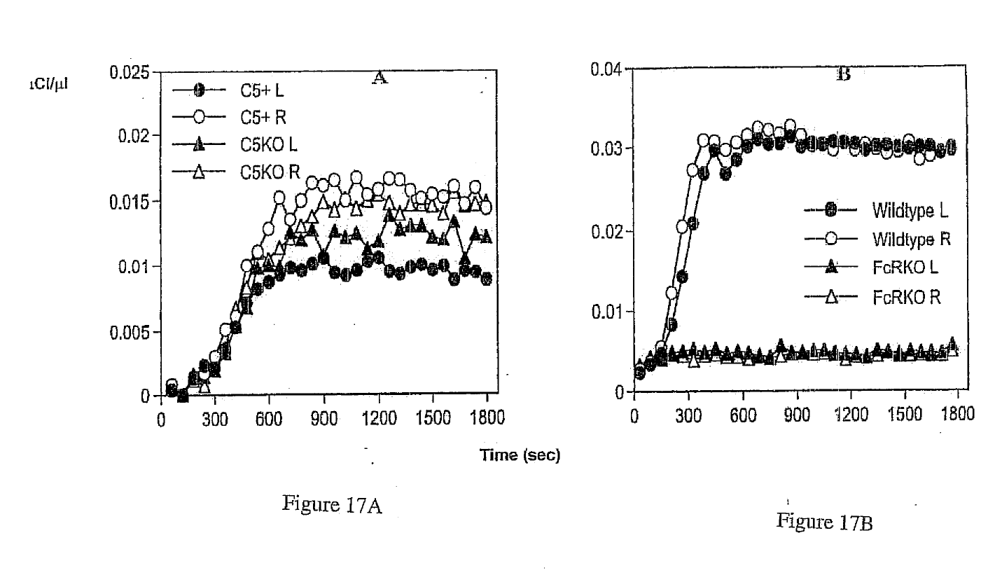

A microPET digital scanning technique is used to quantitate the radioactivity concentration within user-defined regions of interest. Quantitation shows that the ⁶⁴Cu radioactivity density in the ankle region of the wild-type control mouse is 8 times higher than that of a FcR knockout mouse (FIG. 17B). FIG. 17A shows microPET digital scanning data of ⁶⁴Cu radioactivity density in the ankle region of a C5+ mouse and a C5 knockout mouse.

Particular inventive conjugates include a therapeutic moiety which is an siRNA directed to a target gene involved with rheumatoid arthritis. In particular, model target genes include the mouse Fc Receptor III (FcRIII) (NM 010188), mouse tumor necrosis factor TNFα (NM 013693) and mouse interlcukin-lp (IL-1β) (M15131). Seven sites for each target gene are selected and corresponding sense and antisense strands are synthesized, annealed and purified. Sense and antisense strands with 3′TT of siRNAs are synthesized with a 3′-amino modification (Qiagen Inc., CA).

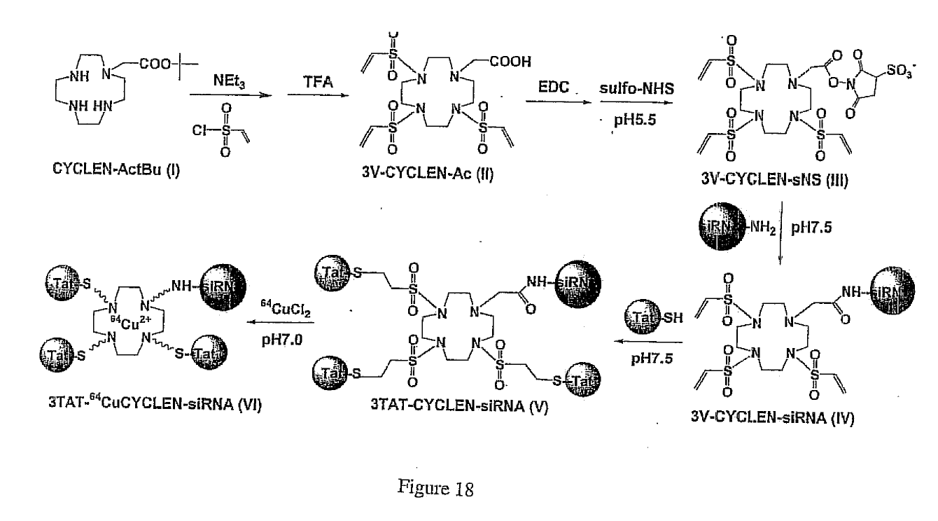

An inventive construct includes a Tat peptide which is synthesized with a modification using a cystine for linkage and a fluorescent reporter for optical imaging (SynPep Inc., CA). In addition a Tat analog (YARAAARQARA) (SEQ ID NO. 46) with reported 33 times more efficient cell transduction than the Tat protein is alternatively used (Ho et al., 2001). A macrocyclic core such as CYCLEN is conjugated to from 1-3 Tat peptides and/or 1-3 Tat analog peptides along with 1 siRNA as a therapeutic agent (see FIG. 18). The macrocyclic core serves not only as a multifunctional linker but also a chelator to bind a radiometal for molecular imaging such as ⁶⁴CU for microPET imaging and ¹¹¹In for microSPECT/CT scanning.

A detailed synthetic scheme for the described inventive conjugate is shown in FIG. 18. Briefly described, the synthetic process involves a protected CYCLEN analog (Macrocyclics, Richardson, Tex.), is first reacted with 3 moles of vinyl sulfone chloride to produce compound II (3V-CYCLEN-Ac). A trifluoroacetic acid treatment removes the protecting groups from the macrocyclic core structure. The compound II is activated by EDC and coupled to sulfo-NHS to give 3V-CYCLEN-sNS (III). An siRNA or other therapeutic molecule of interest is coupled with the 3V-CYCLEN-sNS (III) to give 3V-CYCLEN-siRNA (IV). A synthetic Tat peptide is coupled with 3V-CYCLEN-siRNA (IV) to give 3TAT-CYCLEN-siRNA (V). The 3TAT-CYCLEN-siRNA conjugates are characterized by mass spectrum (MALDI-MS). The concentration of the conjugate is determined by UV spectrophotometry.

### Example 23

Radiolabeling of Tat-CYCLEN-siRNA with ⁶⁴Cu via CYCLEN macrocyclic ligand and characterization of the ⁶⁴Cu-Tat-CYCLEN-siRNA (FIG. 18, VI).

⁶⁴Cu (⁶⁴CuCl₂ in 0.1 M HCl, radionuclide purity greater than 99%) is purchased from MIR, Washington University Medical School. The Tat-CYCLEN-siRNA conjugate is incubated with ⁶⁴Cu in 50 millimolar ammonium citrate buffer, pH 7.0, at 43° Centigrade for 1 hour. The reaction is terminated by addition of a 10 millimolar DTPA solution. Labeled ⁶⁴Cu-Tat-CYCLEN-siRNA is separated by size exclusion Bio-Spin 6 column or a YM-3 Centricon. The efficiency of radiolabeling is measured by integrating areas on the Fast Protein Liquid Chromatography (FPLC) and determining the percentage of radioactivity associated with the ⁶⁴Cu-Tat-CYCLEN-siRNA peaks. ¹¹¹In, with a half-life of 67.9 hours, is obtained from NEN Life Science Products Inc., Boston, Mass. Tat-CYCLEN-siRNA labeling with ₁₁₁In is performed by adding ¹¹¹InCl₃ to Tat-CYCLEN-siRNA in a 50 millimolar glycine/HCl buffer, pH 3.5 at 50° Centigrade for 3 hours. The chelation efficiency is determined by FPLC with a radioactivity detector.

### Example 24

Evaluation of cellular transduction and validation screen of Tat-siRNAs in vitro.

A Tat-CYCLEN-siRNA conjugate having an siRNA directed toward GAPDH (glyceraldehyde 3-phosphate dehydrogenase gene) is measured using the ⁶⁴C labeled Tat-CYCLEN-siRNAs. HeLa cells are incubated with various amounts of Tat-⁶⁴Cu CYCLEN-siRNA in RPMI medium at 37° Centigrade for 24 hours in order to measure the cellular uptake at different times. After incubation cells are washed 3 times with Hanks buffered saline solution and resuspended in RPMI medium to measure the cellular washout effect. In parallel, ⁶⁴Cu CYCLEN-siRNA without Tat peptide is used as a control. Intracellular Tat-⁶⁴Cu CYCLEN-siRNA retention is determined by radioactivity counting.

Macrophage cells are cultured in 96-well plates and induced with lipopolysaccharide (LPS) to induce a high level of expression of TNFα and IL-1β. Transport into cells is evaluated by fluorescent microscopy and fluorescent intensity assay at different times of incubation. Suppression of the siRNA target genes FcRIII, INFα and IL-1β are measured by reverse transcription polymerase chain reaction (RT-PCR), immunostaining, and Western blot techniques. Liposomal transfection agents such Oligofectamine or TransIT-TKO are used as positive siRNA delivery controls. In addition a validated mouse NAPDH siRNA (Qiagen, CO) is used as a positive gene silencing control.

### Example 25

Direct molecular imaging of in vivo trafficking and biodistribution of Tat-⁶⁴Cu CYCLEN-siRNA in the K/Bx and rheumatoid arthritis model.

Balb/c mice are injected by the tail vein route with 250 micrograms of nonlabeled anti-GPI to induce rheumatoid arthritis. At day 0, day 1, day 2 and day 4 after inoculation, 350 microcuries/50 micrograms ⁶⁴Cu labeled biotracer, Tat-⁶⁴Cu-CYCLEN-siRNA, is injected and scanned over a period of 0-60 minutes and at later time points such as 24 hours. In addition, ¹¹¹In labeled Tat-CYCLEN-siRNA conjugates are also selected for microSPECT/CT imaging at 1 hour, 24 hours, 48 hours and 96 hour time points to take advantage of its long half-life.

### Example 26

Functional molecular imaging with nonlabeled Tat-CYCLEN-siRNA and labeled ⁶⁴C-anti-GPI.

The gene silencing effect in vivo of the various constructs in the K/Bx in rheumatoid arthritis model is further characterized by injection of Balb/c mice via the tail vein route with 50 micrograms of nonlabeled Tat-CYCLEN-siRNA conjugates. At days 0, 1, 2, and 4 after injection of the nonlabeled constructs 350 microcuries/250 micrograms ⁶⁴Cu/anti-GPI is injected and scanned from 0-60 minutes and at 24 hours after injection to image the localization of the anti-GPI radiolabeled molecule in distal joints. The knockdown of the genes targeted by the inventive conjugates may be correlated with the reduced effect of the anti-GPI rheumatoid arthritis induction stimulus.

### Example 27

The evaluation of in vivo delivery and gene knockdown efficacy by molecular biology measurements.

Anti-FcRIII, TNFα or IL-1β siRNA conjugates described above are injected in mice by the tail vein route as described in previous examples. Production of TNFα and IL-1β is assessed at various times after injection by ELISA. Further, gene expression of FcRIII, TNFα and IL-1β is assessed at front and rear limbs, knees, and lymph nodes by RT-PCR and Northern blot, Western blot, and immunostaining in peritoneal cells at various times after injection.

### Example 28

Biodistribution study and quantitative autoradiography.

Tat-⁶⁴Cu-CYCLEN-RNA localization is analyzed by traditional biodistribution study immediately after the above-described microPET imaging experiments. In particular, front and rear limbs, knees, liver, and kidney are collected, weighed and activity counted on a gamma counter. The percent injected dose per gram (% ID/g) for each tissue and organ is calculated and compared with the quantitative microPET imaging data.

Whole body quantitative autoradiography (QAR, resolution 0.1 mm) is performed on 1 mouse for each radiotracer. The mouse is frozen in isopropanol-dry ice mixture and 50 micron coronal sections are obtained with a Hacker-Bright 5030/WD/MR cryomicrotome (Hacker Instruments, Fairfield, N.J.). Phosphor screens and tissue slices are loaded into film cassettes and stored in a −20° Centigrade freezer. Images are developed using an automated phosphor storage system (Packard Instruments cyclone model) and calibrated by an internal standard. Time of sacrifice for QAR matches times of the radiolabel as determined in the above-described microPET experiment. Both microPET and QAR quantification are normalized as necessary.

### Example 29

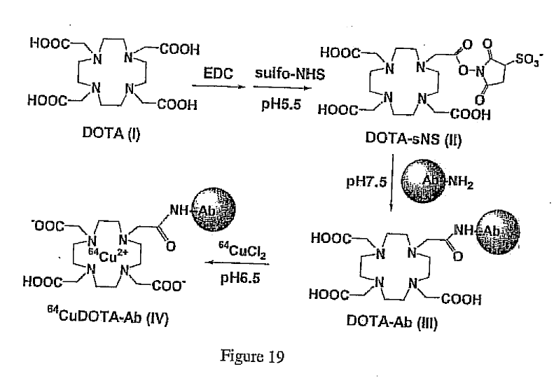

A schematic illustration of preparation and radiolabeling of DOTA-anti-GPI conjugate with ⁶⁴Cu is shown in FIG. 19. 1,4,7,10-tetraazacyclododecane-N,N′, N″,N′″-tetraacetic acid (DOTA) is activated by 1-ethyl-3-[3-(dimethylamino)propyl]carbodiimide (EDC) and reacted with N-hydroxysulfosuccinimide (sulfo-NHS) in a mixture solution of pH 5.5 at 4° Centigrade for 30 minutes. Purified anti-GPI antibodies are reacted with a 1000;1000:100:1 molar ratio of DOTA:sulfo-NHS:EDC antibody in 0.1 M Na₂HPO₄ buffer of pH 7.5 at 4° Centigrade for 12-16 hours. After conjugation the reaction mixture is centrifuged repeatedly through a YM-30 Centricon with 50 millimolar ammonium citrate buffer of pH 6.5 in order to remove small molecules.

### Example 30

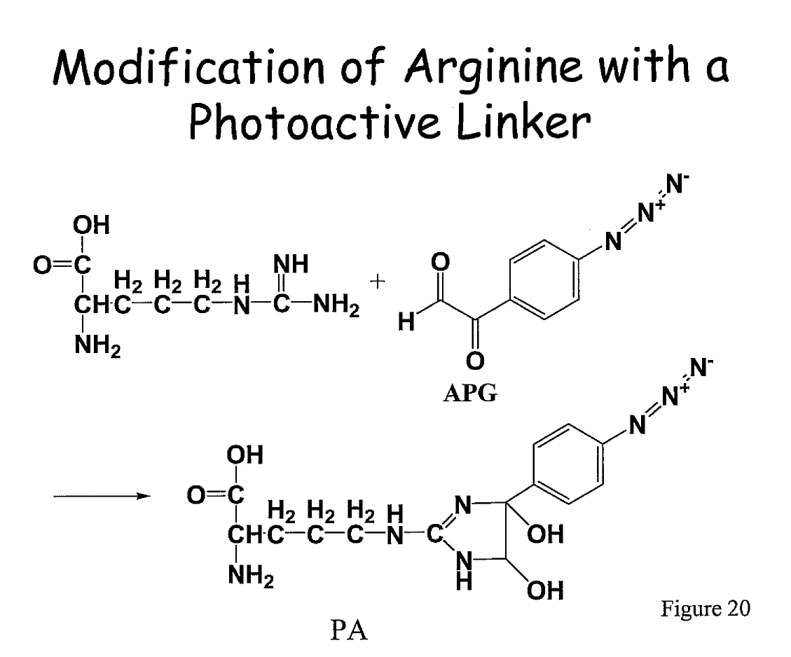

A photoactive linker is used in one example to link a cargo moiety to a scaffold. FIG. 20 shows a scheme for modification of an arginine with a photoactive linker, p-azidophenyl glyoxal (APG) which is reactive with amino groups and the guanidium side chain of arginine.

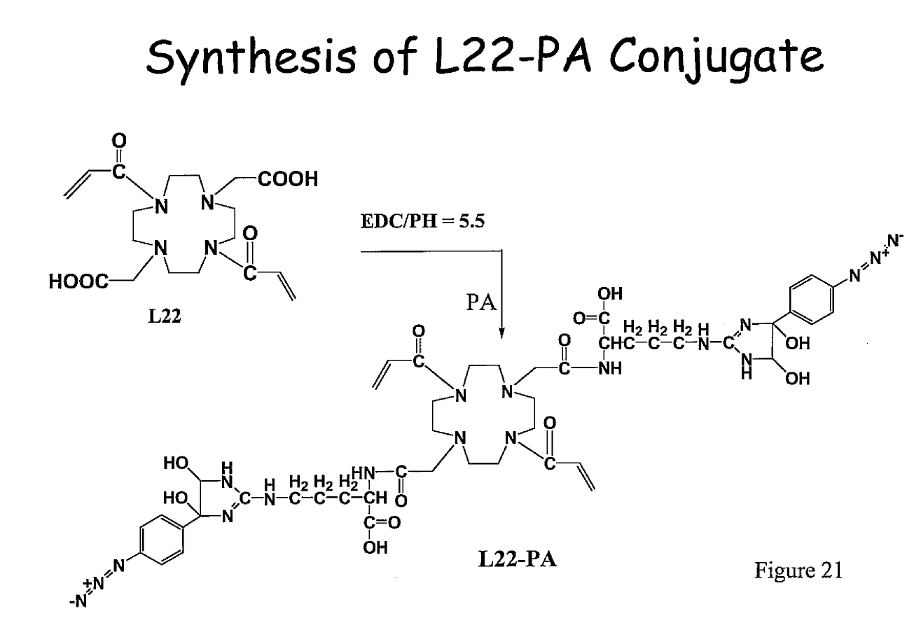

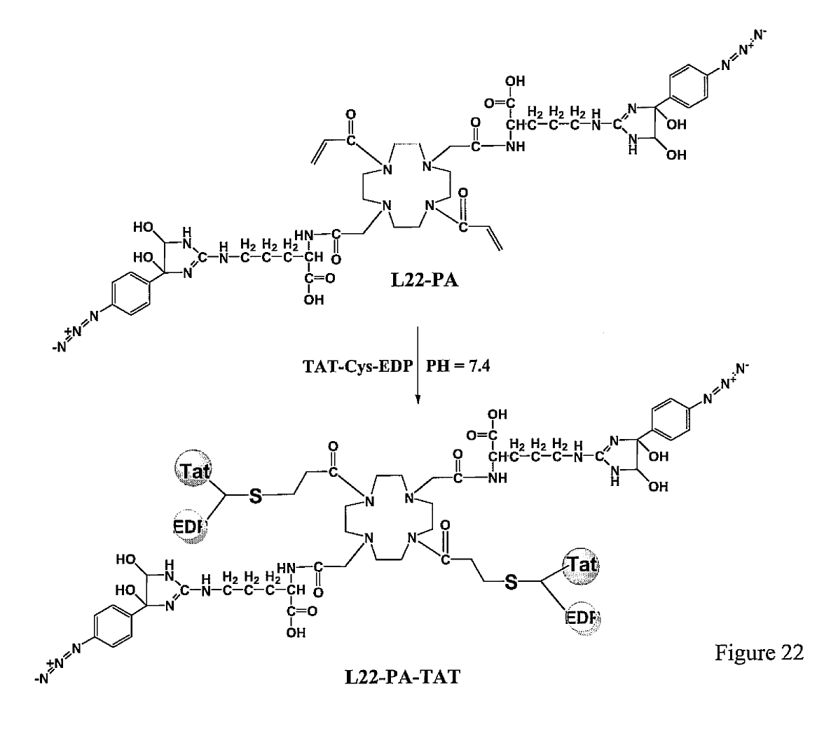

FIG. 21 illustrates a synthetic scheme for addition of an arginine-photoactive linker complex, as shown in FIG. 22, to a tetrazamacrocycle scaffold derivative. The scaffold derivative moiety and arginine-photoactive linker complex are reacted in the presence of a water-soluble carbodiimide coupler, EDC, at pH 5.5, to yield the depicted compound.

FIG. 22 illustrates conjugation of a translocating moiety, Tat, and an endosome disruption moiety, EDP to yield the conjugate shown.

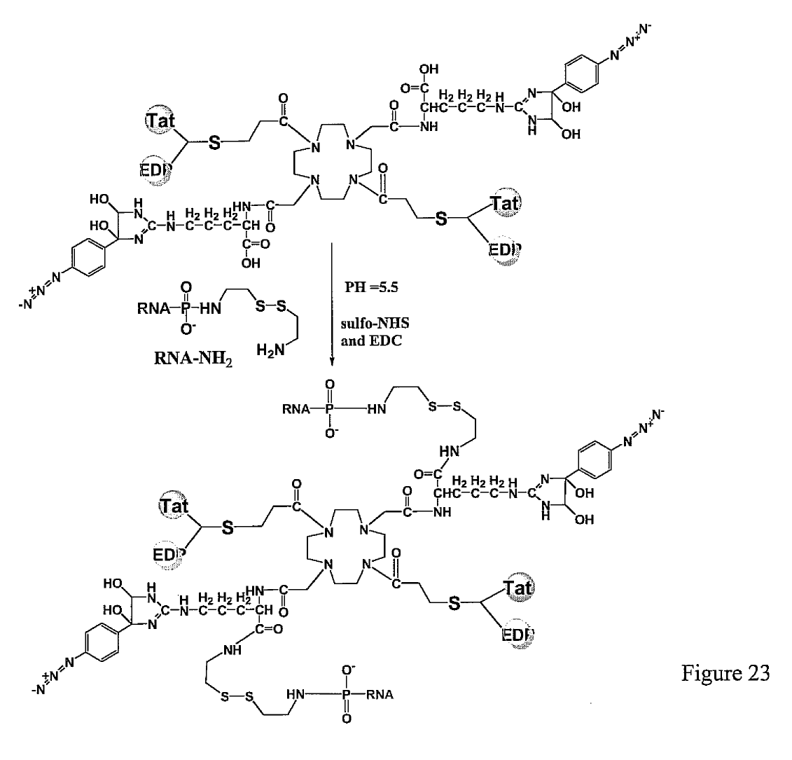

FIG. 23 further shows a scheme for addition of an oligonucleotide to a conjugate, in the presence of EDC and sulfo-NHS at pH 5.5 to yield a multifunctional conjugate.

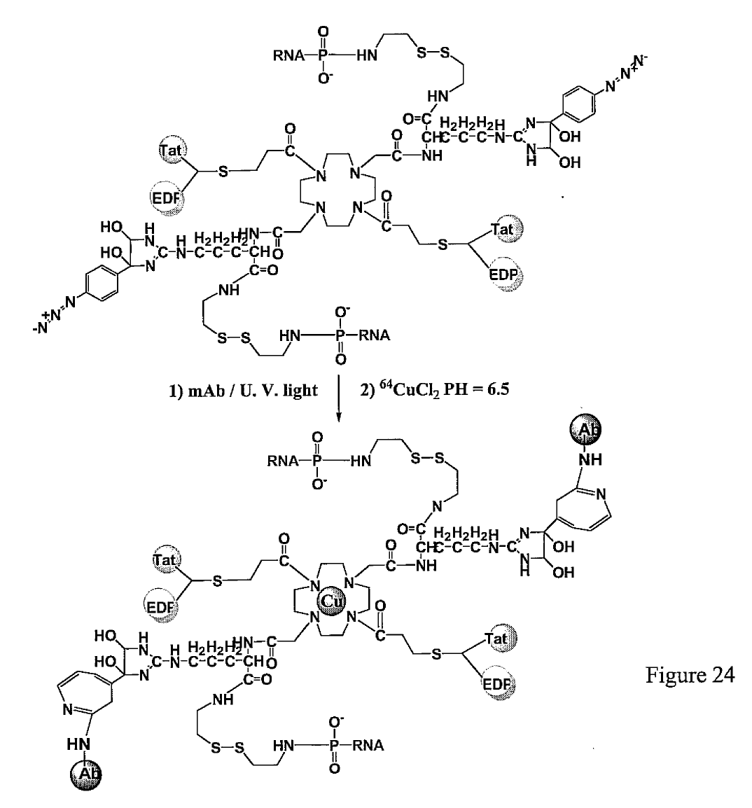

FIG. 24 shows a method of addition of an antibody and a radionuclide to a conjugate. In this example, a conjugate having a photoactive linker is incubated with an antibody of interest in the presence of UV light, yielding an antibody conjugate. Further, the conjugate is incubated with ⁶⁴CuCl₂ at pH 6.5 such that a conjugate chelate is formed as depicted in FIG. 24.

### References

1. O'Driscoll, L., Linehan, R. & Clynes, M. Survivin: role in normal cells and in pathological conditions. Curr. Cancer Drug Targets. 3, 131-152 (2003).
2. Fukuda, S. & Pelus, L. M. Regulation of the inhibitor-of-apoptosis family member survivin in normal cord blood and bone marrow CD34(+) cells by hematopoietic growth factors: implication of surviving expression in normal hematopoiesis. Blood 98, 2091-2100 (2001).
3. Fukuda, S. & Pelus, L. M. Elevation of Survivin Levels by hematopoietic growth factors occurs in quiescent CD 34+ hematopoictic stem and progenitor cells before cell cycle entry. Cell Cycle 1, 322-326 (2002).
4. Gianani R. et al. Expression of survivin in normal, byperplastic, and neoplastic colonic mucosa. Hum. Pathol. 32, 119-125 (2001).
5. Kobayashi, Y. et al. Developmentally regulated expression of survivin in the human thymus. Hum. Immunol. 63, 101-107 (2002).
6. Kornacker, M., Vernersis, M. R., Komacker, B., Scheffold, C. & Negrin, R. S. Survivin expression correlates with apoptosis resistance after lymphocyte activation and is found preferentially in memory T Cells. Immunol. Lett. 76, 169-173 (2001).
7. O' Connor, D. S. et al. Control of apoptosis during antiogenesis by survivin expression in endothelial cells. Am, J. Pathol. 156, 393-398 (2000).
8. Dong, Y., Sui, L., Watanabe, Y., Sugimoto, K. & Tokuda, M. Survivin expression in laryngeal squamous cell carcinomas and its prognostic implications. Anticancer Res. 22, 2377-2383 (2002).
9. Kajiwara, Y. et al. Expression of survivin in astrocytic tumors: correlation with malignant grade and prognosis. Cancer 97, 1077-1083 (2003).
10. Rodel, F. et al. High survivin expression is associated with reduced apoptosis in rectal cancer and may predict disease-free survival after preoperative radiochernotherapy and surgical resection. Strahlenther. Onkol. 178, 426-435 (2002).
11. Ikeguchi, J., Ueda, T., Sakatani, T., Hirooka, Y. & Kaibara, N. Expression of survivin messenger RNA correlates with poor prognosis in patients with hepatocellular carcinoma. Diagn. Mol. Pathol. 11, 33-40 (2002).
12. Schlette, E. J., Medeiros, L. J., Goy, A., Lai, R. & Rassidakis, G. Z. Survivin expression predicts poorer prognosis in anaplastic large-cell lymphoma. J. Clin. Oncol 22, 1682-1688 (2004),
13. Tamm, I. et al. High expression levels of x-linked inhibitor of apoptosis protein and survivin correlate with poor overall survival in childhood de novo acute mycloid leukemia. Clin. Cancer Res. 10, 3737-3744 (2004).
14. Wang, L., Zhang, G. M. & Feng, Z. H. Down-regulation of survivin expression reversed multidrug resistance in adriamycin-resistant HL-60/ADR cell line. Acta Pharmacol. Sin. 24, 1235-1240 (2003).
15. Zaffaroni, N. & Daidone, M. G. Survivin expression and resistance to anticancer treatments: perspectives for new therapeutic interventions. Drug Resis, Updat. 5, 65-72 (2002).
16. Li. Y. H., Wang, C., Meng, K., Chen, L. B. & Zhou, X. J. Influence of survivin and caspase-3 on cell apoptosis and prognosis in gastric carcinoma. World J. Gastroenterol. 10, 1984-1988 (2004).
17. Asanuma, K. et al. Survivin as a radioresistance factor in pancreatic cancer. Jpn. J. Cancer Res. 91, 1204-1209 (2000).
18 Lu, B. et al. Survivin as a therapeutic target for radiation sensitization in lung cancer. Cancer Res, 64, 2840-2845 (2004).
19. Kim, S. et al. Phosphatidylinositol 3-Kinase inhibition down-regulates survivin and facilitates TRAIL-mediated apoptosis in neuroblastomas. J. Pediatr. Surg. 39, 516-521 (2004).
20. Griffith, T. S. et al. Induction and regulation of tumor necrosis factor-related apoptosis-inducing ligand/Apo-2 ligand-mediated apoptosis in renal cell carcinoma. Cancer Res. 62, 3093-3099 (2002).
21. Li, T., Brouha, B. & Grossman, D. Rapid induction of mitochondrial events and caspase-independent apoptosis in Survivin-targeted melanoma cells. Oncogene 23, 39-48 (2004).
22. Shin, S. et al. An Anti-apoptotic protein human survivin is a direct inhibitor of caspase-3 and -7. Biochemistry 40, 1117-1123 (2001).
23. Banks, D. P. et al. Survivin does not inhibit caspase-3 activity. Blood 96, 4002-4003 (2000).
24. Chandele, A., Prasad, V., Jagtap, J. C., Shukla, R. & Shastry, P. R. Upregulation of survivin in G2/M cells and inhibition of caspase 9 activity enhances resistance in staurosporine-induced apoptosis. Neoplasia 6, 29-40 (2004).
25. Dohi, T. et al. An 1AP-1AP Complex Inhibits Apoptosis. J. Biol. Chem. 279, 34087-34090 (2004).
26. Song, Z., Yao, X. & Wu, M. Direct interaction between survivin and Smac/DLABLO is essential for the anti-apoptotic activity of survivin during taxol-induced apoptosis. J. Biol. Chem. 278, 23130-23140 (2003).
27. Fukuda, S., Mantel, C. R. & Pelus, L. M. Survivin regulates hematopoietic progenitor cell proliferation through p21WAF1/Cip1-dependent and -independent pathways. Blood 103, 120-127 (2004).
28. Suzuki A. et al. Survivin initiates procaspase 3/p21 complex formation as a result of interaction with Cdk4 to resist Fas-mediated cell death. Oncogene 19, 1346-1353 (2000).
29. Beltrami, E., Plescia, J., Wilkinson, J. C., Duckett, C. S. & Altieri, D. C. Acute ablation of survivin uncovers p53-dependent mitotic checkpoint functions and control of mitochondrial apoptosis. J. Biol. Chem. 279, 2077-2084 (2004).
30. Kikita S., et al. Overexpression of TIAP/m-survivin in thymocytes enhances cell proliferation. Mol. Immunol. 39, 289-298 (2002).
31. Fukuda, S., Foster, R. G., Porter, S. B. & Pelus, L. M. The antiapoptosis protein survivin is associated with cell cycle entry of normal cord blood CD34(+) cells and modulates cell cycle and proliferation of mouse hematopoietic progenitor cells. Blood 100, 2463-2471 (2002).
32. Li. F. et al. Peliotropic cell-division defects and apoptosis induced by interference with survivin function. Nat. Cell Biol. 1, 461-466 (1999).
33. Beardmore, V. A., Ahonen, L. J., Gorbsky, G. J. & Kalio, M. J. Survivin dynamics increases at centromeres during G2/M phase transition ad is regulated by microtubule-attachment and Aurora B kinase activity. J. Cell Sci. 117, 4033-4042 (2004).
34. Chen, J. et al. Survivin enhances Aurora-B kinase activity and localizes Aurora-B in human cells. J. Biol. Chem. 278, 486-490 (2003).
35. Bolton, J. A. et al. Aurora B kinase exists in a complex with survivin and INCENP and its kinase activity is stimulated by survivin binding and phosphorylation. Mol. Biol. Cell 13, 3064-3077 (2002).
36. Wheatley, S. P., Carvalho, A., Vagnarelli, P. & Earnshaw, W. C. INCENP is required for proper targeting of Survivin to the centromeres and the anaphase spindle during mitosis. Curr. Biol. 11, 886-890 (2001).
37. Tran, J. et al. Marked induction of the LAP family antiapoptotic proteins survivin and X1AP by VEGF in vascular endothelial cells. Biochem. Biophys. Res. Commun. 264, 781-788 (1999).
38. Papapetropoulos, A. et al. Angiopoietin-1 inhibits endothelial cell apoptosis via the Akt/survivin pathway. J. Biol. Chem. 275, 9102-9105 (2000).
39. Blanc-Brude, O. P. et al. Therapeutic targeting of the survivin pathway in cancer: initiation of mitrochondrial apoptosis and suppression of tumor-associated angiogenesis. Clin. Cancer Res. 9, 2683-2692 (2003).
40. Carter, B. Z. et al. Targeting Survivin expression induces cell proliferation defect and subsequent cell death involving mitochondrial pathway in myeloid leukemic cells. Cell Cycle 2, 488-493 (2003).
41. Coma, S. et al. Use of siRNAs and Antisense Oligonucleotides Against Survivin RNA to Inhibit Steps Leading to Tumor Angiogenesis. Oligonucleotides 14, 100-113 (2004).
42. Olie, R. A. et al. A novel antisense oligonucleotide targeting survivin expression induces apoptosis and sensitizes lung cancer cells to chemotherapy. Cancer Res. 60, 2805-2809 (2000).
43. Tu, S. P. et al. Suppression of survivin expression inhibits in vivo tumorigenicity and angiogenesis in gastric cancer. Cancer Res. 63, 7724-7732 (2003).
44. Grossman, D., McNiff, J. M., Li, F. & Altieri, D. C. Expression and targeting of the apoptosis inhibitor, survivin, in human melanoma. J. Invest Dermatol. 113, 1076-1081 (1999).
45. Pennati, M. et al. Ribozyrne-mediated down-regulation of survivin expression sensitizes human melanoma cells to topotecan in vitro and in vivo. Carcinogenesis 25, 1129-1136 (2004).
46. Pennati, M. et al. Ribozyne-mediated inhibition of survivin expression increases spontaneous and drug-induced apoptosis and decreases the tumorigenic potential of human prostate cancer cells, Onocogene 23, 386-394 (2004).
47. Uchida, H. et al. Adenovirus-mediated transfer of siRNA against survivin induced apoptosis and attenuated tumor cell growth in vitro and in vivo. Mol. Ther. 10, 162-171 (2004).
48. Ling, X., Bemacki, R. J., Brattain, M. G. & Li, F. Induction of surivin expression by taxol (paclitaxel) is an early event, which is independent of taxol-mediated G2/M arrest. J. Biol. Chem. 279, 15196-15203 (2004).
49. ICurreck, J. Antisense technologies. Improvement through novel chemical modifications. Eur. J. Biochem. 270, 1628-1644 (2003).
50. Minakuchi, Y. et al. Atelocollagen-mediated synthetic small interfering RNA delivery for effective gene silencing in vitro and in vivo. Nucleic Acids Res. 32, el09 (2004).
51. Grunweller, A. et al. Comparison of different antisense strategies in mammalian cells using locked nucleic acids, 2′-O-methyl RNA, phosphorothioates and small interfering RNA. Nucleic Acids Res. 31, 3185-3193 (2003).
52. Layzer, J. M. et al. In vivo activity of nuclease-resistant siRNAs. RNA 10, 766-771 (2004).
53. Schwarze, S. R. & Dowdy, S. F. In vivo protein transduction: intracellular delivery of biologically active proteins, compounds and DNA. Trends Pharmacol. Sci. 21, 45-48 (2000).
54. Ho, A., Schwarze, S. R., Mermelstein, S. J., Waksman, G. & Dowdy, S. F. Synthetic protein transduction domains: enhanced transduction potential in vitro and in vivo, Cancer Res. 61, 474-477 (2001).
55. Jarver, P. & Langel, U. The use of cell-penetrating peptides as a tool for gene regulation. Drug Discov. Today 9, 395-402 (2004).
56. Gallazzi, F. et al. Synthesis of radiometal-labeled and fluorescent cell-permeating peptide-PNA conjugates for targeting the bcl-2 proto-oncogene. Bioconjug. Chem. 14, 1083-1095 (2003).
57. Lewis, M. R. et al. Radiometal-labeled peptide-PNA conjugates for targeting bcl-2 expression: preparation, characterization, and in vitro mRNA binding. Bioconjug. Chem. 13, 1176-1180 (2002).
58. Astriab-Fisher, A., Sergueev, D. S., Fisher M.,- Shaw, B. R. & Juliano, R. L. Antisense inhibition of P-glycoprotein expression using peptide-oligonucleotide conjugates. Biochem. Pharmacol. 60, 83-90 (2000).
59. Hemmings-Mieszczak, M., Dom, G., Natt, F. J., Hall, J. & Wishart, W. L. Independent combinatorial effect of antisense oligonucleotides and RNAI-mediated specific inhibition of the recombinant rat P2X3 receptor. Nucleic Acids Res. 31, 2117-2126 (2003).
60. Jiang, X. et al. Participation of Survivin in mitotic and apoptotic activities of normal and tumor-derived cells. J. Cell Biochem. 83, 342-354 (2001).
61. Cho, Y. W., Kim, J. D. & Park, K. Polycation gene delivery systems: escape from endosomes to cytosol. J. Pharm. Pharmacol. 5, 721-734 (2003).
62. Giodini, A. et al. Regulation of microtubule stability and mitotic progression by survivin. Cancer Res. 62, 2462-2467 (2002).
63. Brummelkamp T. R., et al. A system for stable expression of short interfering RNAs in mammalian cells. Science 296, 550-3 (2002).
64. Eguchi A., Akuta T., Okuyama H. et al. Protein transduction domain of HIV-1 Tat protein promotes efficient delivery of DNA into mammalian cells. J. Biol. Chem. 276 (28), 26204-10 (2001).
65. Gambhir S. S., Barrio J. R., Herschman H. R., Phelps M. E. Imaging gene expression: principles and assays. J. Nucl. Cardiol. 6, 219-233 (1999).
66. Hnatowich D. J. Antisense and nuclear medicine, J. Nucl. Med. 40, 693-703 (1999).
67. Ho A., Schwarze S. R., Wemelstein S. J., Waksman G., Dowdy S. F. Synthetic protein transduction domains: enhanced transduction potential in vitro and in vivo. Cancer Res. 61, 474-477 (2001).
68. Jiang M., Milner J. Selective silencing of viral gene expression in HPV-positive human cervical carcinoma cells treated with siRNA, a prinmer of RNA interference. Oncogene 21 (39), 6041-8 (2002).
69. Kim V. N. RNA Interference in Functional Genomics and Medicine. J. Korean Med. Sci. 18, 309-18 (2003).
70. McManus M. T., Sharp P. A. Gene silencing in mammals by small interfering RNAs. Nat. Rev. Genet. 3 (10), 737-47 (2002).
71. Miller V. M., Xia H., Marrs G. L., Gouvion C. M., Lee G., Davidson B. L., Paulson H. L. Allele-specific silencing of dominant disease genes. Proc. Natl. Acad. Sci. 100 (12), 7195-200 (2003).
72. Phelps M. Commentary: nuclear medicine, molecular imaging, and molecular medicine. J. Nucl. Med. 43, 13N-15N (2002).
73. Qin X. F., An D. S., Chen I. S., Baltimore D. Inhibiting HIV-1 infection in human T cells by lentiviral-mediated delivery of small interfering RNA against CCR5. Proc, Natl. Acad, Sci. 100 (1), 183-8 (2003).
74. Randall, G., Grakoui A., Rice C. M. Clearance of replicating hepatitis C virus replicon RNAs in cell culture by small interfering RNAs. Proc. Natl. Acad. Sci. 100 (1), 235-40 (2003).
75. Robbins P. D., Evans C. H., Chemajovsky Y. Gene therapy for arthritis. Gene Therapy 10 (10), 902-11 (2003).
76. Rubinson D. A., Dillon C. P., Kwiakowski A. V., Sievers C., Yang L., Kopinja J., Rooney D. L., Ihrig M. M., McManus M. T., Gertler F. B., Scott M. L., Van Parijs L. A lentivirus-based system to functiontionally silence genes in primary mammalian cells, stem cells and transgenic mice by RNA interference. Nat. Genet. 33 (3), 401-6 (2003).
77. Rudolph C., Plank C., Lausier J., Schillinger U., Muller R. H., Rosenecker J. Oligomers of the arginine-rich motif of the HIV-1 Tat protein are capable of transferring plasmid DNA into cells. J. Biol. Chem. 278 (13), 11411-8 (2003).
78. Schwarze S. R., Ho A., Vocero-Akbani A., Dowdy S. F. In vivo protein transduction: delivery of a biologically active protein into the mouse. Science 285, 1569-72 (1999).
79. Schwarze, S. R., Dowdy S. F. In vivo protein transduction: intracellular delivery of biologically active proteins, compounds and DNA. Trends Pharmacol. Science 21(2), 45-8 (2000).
80. Shlomai A., Shaul Y. Inhibition of hepatitis B virus expression and replication by RNA interference. Hepatology 37 (4), 764-70 (2003).
81. Simeoni F., Morris M. C., Heitz F., Divita G., Insight into the mechanism of the peptide-based gene delivery system MPG: implications for delivery of siRNA into mammalian cells. Nucleic Acids Res. 31 (11), 2717-24 (2003).
82. Sorenson D. R., Leirdal M., Sioud M. Gene silencing by systemic delivery of synthetic siRNAs in adult mice. J. Mol. Biol. 327 (4), 761-6 (2003).
83. Shuey D. J., McCallus D. E., Giordano T. RNAi: gene-silencing in therapeutic intervention. Drug Discov. Today 7 (20), 1040-6 (2002).
84. Torchilin V. P., Levchenko T. S., Rammohan R., Volodina N., Papahadjopoulos-Sternberg B., D'Souza G. G Cell transfection in vitro and in vivo with nontoxic Tat peptide-liposome-DNA complexes. Proc. Natl. Acad. Sci. 100 (4), 1972-7 (2003).
85. Wipke B. T., Wang Z., Kim J., McCarthy J. T., Allen P. M. Dynamic visualization of the rapid initiation of a joint-specific autoimmune response through positron emission tomography. Nature Immunology 3, 366-372 (2002).
86. Wilson J. A., Jayasena S., Khvorova A., Sabatinos S., Rodrigue-Gervais I. G., Arya S., Sarangi F., Harris-Brandts M., Beaulieu S., Richardson C. D. RNA interference blocks gene expression and RNA synthesis from hepatitis C replicons propagated in human liver cells. Proc. Natl. Acad. Sci. 100 (5), 2783-8 (2003).
87. Zhang Y. M., Wang Y., Liu N., Zhu Z. H., Rusckowski M+, Hnatowich D. J. In vitro investigations of tumor targeting with Tc-99m labeled antisense DNA. J. Nucl. Med. 42, 1660-1669 (2001).

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A conjugate composition, comprising:
a scaffolding specie comprising an azamacrocycle conjugated to at least one cargo specie and at least one trafficking specie.

2. The conjugate composition of claim 1 wherein said at least one cargo specie is a therapeutic moiety and said at least one trafficking specie comprises a cellular targeting moiety and a translocating moiety.

3. The conjugate composition of claim 1 wherein said at least one cargo specie is a therapeutic moiety and said at least one trafficking specie comprises a cellular targeting moiety and an endosome disruption moiety.

4. The conjugate composition of claim 2 further comprising a reporter moiety.

5. The conjugate composition of claim 1 wherein said at least one cargo specie is a therapeutic moiety and said at least one trafficking specie comprises a cellular targeting moiety and an organelle targeting moiety.

6. The conjugate composition of claim 3 further comprising a translocating moiety.

7. The conjugate composition of claim 2 further comprising a second translocating moiety.

8. The composition of claim 2 further comprising a second endosome disruption moiety.

9. The composition of claim 2 further comprising a second therapeutic moiety.

10. The composition of claim 1 wherein the cargo specie is conjugated to the scaffold specie by a linkage to a nitrogen member of said azamacrocycle specie.

11. The composition of claim 9 wherein said linkage to the nitrogen member of the azamacrocycle is a bond selected from the group N—C, N—S, N—O and N—N.

12. The composition of claim 10 wherein a plurality of cargo species, trafficking species, or a combination thereof are coupled to said scaffolding specie through said linkage.

13. The composition of claim 1 wherein a disulfide bond is intermediate between said cargo specie and said scaffolding specie.

14. The composition of claim 1 wherein the azamacrocycle has at least three nitrogen members.

15. The composition of claim 14 wherein the azamacrocycle is selected from the group consisting of: oxo-aza crown ethers, thia-azo crown ethers, aza-thia-oxo crown ethers, cryptands, cavitands, podands, corrinoids, porphyrins, benzophyrins, porphycenes, chlorines, benzochlorins and purpurins.

16. The composition of claim 4 wherein said reporter moiety is a metal ion.

17. The composition of claim 1 further comprising an organic macrocycle coupled to one of said scaffolding specie, said cargo specie and said trafficking specie.

18. A pharmaceutical composition comprising:
a conjugate according to claim 1; and
a pharmaceutically acceptable carrier.

19. A method of medical treatment, comprising: administering a therapeutically effective amount of a pharmaceutical composition according to claim 18.

20. A method of imaging a cell comprising:
exposing said cell to a conjugate composition comprising a scaffolding specie comprising an azamacrocycle conjugated to one translocating moiety, a cargo specie capable of binding an intracellular component of said cell and a reporter moiety;
allowing sufficient time for said conjugate to enter said cell; and
imaging said cell to detect a signal indicative of said reporter moiety.

21. The method of claim 20 wherein said cargo specie binds survivin.

22. The method of claim 21 wherein said cargo specie is an antisense oligonucleotide.


SEQUENCE LISTING (38 sequences)

SEQ ID NO: 1 - protein, 10 aa, Human immunodeficiency virus type 1 <s300> Schwarze SR et al., In vivo protein transduction intracellular delivery of biologically active proteins, compounds and DNA Trends Pharmacol. Sci. 21 45-48 2000
GRKKRRQRRR

SEQ ID NO: 2 - protein, 11 aa, Human immunodeficiency virus type 1 <s300> Turner et al., Nucleic Acids Research 33 27-42 2005
GRKKRRQRRRP

SEQ ID NO: 3 - protein, 11 aa, Artificial
  a Tat analog as a translocating moiety <s300> Ho, A. et al. Synthetic protein transduction domain enhanced transduction potential in vitro and in vivo Cancer Research 61 474-477 2001
YARAAARQARA

SEQ ID NO: 4 - protein, 18 aa, Human immunodeficiency virus type 1 <s300> Turner et al., Nucleic Acids Research 33 27-42 2005
RQIKIWFQNRRMKWKKGG

SEQ ID NO: 5 - protein, 27 aa, Human immunodeficiency virus type 1 <s300> Turner et al., Nucleic Acids Research 33 27-42 2005
GWTLNSAGYLLGKINLKALAALAKKIL

SEQ ID NO: 6 - protein, 10 aa, Herpex simplex virus-1 <s300> Hearn AR et al., Trafficking of exogenous peptides into proteasome- dependent major histocompatibility complex class I pathway following enterotoxin B subunit-mediated delivery Journal of Biological Chemistry 279 49 51315-51322 2004-09-01
AVGAGATAEE

SEQ ID NO: 7 - protein, 25 aa, Vesicular stomatitis Indiana virus <s300> Bilsel PA et al., Polymerase errors accumulating during natural evolution of the glycoprotein gene fo vesicular stomatitis virus Indiana serotype isolates Journal of Virology 64 10 4873-4883 1990
KFTIVFPHNQKGNWKNVPSNYHYCP

SEQ ID NO: 8 - protein, 58 aa, Radiation murine leukemia virus <s300> Poliquin L et al., Determinations of thymotropism in Kaplan radiation leukemia virus and nucleotide sequence of its envelope region Journal of Virology 66 8 5141-5146 1992
EPVSLTLALLLGGLTMGGIAAGVGTGTTALVATQQAVGIGALFLGFLGAAGSTMGARS

SEQ ID NO: 9 - protein, 17 aa, Human immunodeficiency virus <s300> Soukchareun et al, Preparation and characterization of antisense oligonucleotide-peptide hybrids containing viral fusion peptides Bioconjugate Chem. 6 43-53 1995
AVGAIGALFLGFLGAAG

SEQ ID NO: 10 - protein, 25 aa, Human immunodeficiency virus <s300> Lear JD et al., Membrane binding and conformational properties of peptides representing the NH2 terminus of influenza HA-2 Journal of Biological Chemistry 262 14 6500-6505 1987-05-15
GLFGAIAGFIENGWEGMIDGWYGFR

SEQ ID NO: 11 - protein, 25 aa, Human immunodeficiency virus <s300> Storm G et al., Functional characterization of an endosome-disruptive peptide and its application in cytosolic delivery of immunoliposome-entrapped proteins Journal of Biological Chemistry 277 30 27135-27143 2002-07-26
GLFGAIAGFIENGWEGMIDGWYGFR

SEQ ID NO: 12 - protein, 23 aa, Human immunodeficiency virus <s300> Nieva et al., Interaction of the HIV-1 fusion peptide with phospholipid vesicles different structural requirements for fusion and leakage Biochemistry 33 3201-3209 1994
AVGIGALFLGFLGAAGSTMGAAS

SEQ ID NO: 13 - protein, 26 aa, Simian virus 41 <s300> Tsurudome M et al., Transcripts of simian virus 41 matrix gene are exclusively dicistronic with the fusion gene which is also transcribed as a monocistron Virology 184 1 93-100 1991
FAGWIGLAALGVATAANVTAAVALVK

SEQ ID NO: 14 - protein, 19 aa, Semliki Forest virus <s300> Santagati MG et al., Multiple repeating motifs are found in the 3′-terminal non-translated region of semliki Forest virus A7 variant genome Journal of General Virology 75 6 1499-1504 1994
KVYTGVYPFMWGGAYCFCD

SEQ ID NO: 15 - protein, 23 aa, Cavia porcellus <s300> Wolfsberg TG et al., The precursor region of a protein active in sperm-egg fusion contains a metalloprotease and a disintegrin domain structural, functional, and evolutionary implications Proc. Natl. Acad. Sci. U.S.A. 90 22 10783-10787 1993
KLICTGISSIPPIRALFAAINIP

SEQ ID NO: 16 - protein, 22 aa, Sendai virus <s300> Tashiro M et al., Characterization of a pantropic variant of Sendai virus derived from a host range mutant Virology 165 2 577-583 1988
FFGAVIGTIALGVATATAAQIT

SEQ ID NO: 17 - protein, 23 aa, Measles virus <s300> Yeagle PL et al., Effects of the ′fusion peptide′ from measles virus on the structure of N-methyl dioleoylphosphatidylethanolamine membranes and their fusion with Sendai virus. Biochimica et Biophysica Acta 1065 49-53 1991
FAGVVIGLAALAGVATATAAQVT

SEQ ID NO: 18 - protein, 22 aa, Newcastle disease virus <s300> Schaper UM et al., Nucleotide sequence of the envelope protein genes of a highly virulent, neurotropic strain of Newcastle disease virus Virology 165 1 291-295 1998
FIGAIIGGVALGVATATAAQIT

SEQ ID NO: 19 - protein, 28 aa, Influenza virus X-31 <s300> Plank et al, The influence of endosome-disruptive peptides on gene transfer using synthetic virus-like gene transfer systems Journal of Biological Chemistry 269 12918-12924 1994
GLFGAIAGFIENGWEGMIDGWYGFRHQN

SEQ ID NO: 20 - protein, 28 aa, Influenza virus X-31 <s300> Plank et al., The influence of endosome-disruptive peptides on gene transfer using synthetic virus-like gene transfer systems Journal of Biological Chemistry 269 12918-12924 1994
GLFGAIAGFIENGWEGLVDGWYGFRHQN

SEQ ID NO: 21 - protein, 29 aa, Influenza virus X-31 <s300> Plank et al., The influence of endosome-disruptive peptides on gene transfer using synthetic virus-like gene transfer systems Journal of Biological Chemistry 269 12918-12924 1994
GFFGAIAGFLEGGWEGMIAGWHGYTSHGB

SEQ ID NO: 22 - protein, 30 aa, Mouse mammary tumor virus <s300> Moore R et al., Complete nucleotide sequence of a milk-transmitted mouse mammary tumor virus two frameshift suppression events are required for translation for gag and pol Journal of Virology 61 2 480-490 1987
FVAAIILGISALIAIITSFAVATTALVKEM

SEQ ID NO: 23 - protein, 23 aa, Influenza virus X-31 <s300> Plank et al., The influence of endosome-disruptive peptides on gene transfer using synthetic virus-like gene transfer systems Journal of Biological Chemistry 269 12918-12924 1994
GLFGAIAGFIENGWEGMIDGGGC

SEQ ID NO: 24 - protein, 23 aa, Influenza virus X-31 <s300> Plank et al., The influence of endosome-disruptive peptides on gene transfer using synthetic virus-like gene transfer systems Journal of Biological Chemistry 269 12918-12924 1994
GLFGAIAGFIENGWEGMIDGWYG

SEQ ID NO: 25 - protein, 34 aa, Influenza virus X-31 <s300> Plank et al., The influence of endosome-disruptive peptides on gene transfer using synthetic virus-like gene transfer systems Journal of Biological Chemistry 269 12918-12924 1994
GLFGAIAGFIENGWEGLAEALAEALEALAAGGSC

SEQ ID NO: 26 - protein, 53 aa, Moloney murine leukemia virus
EPVSLTLALLLGGLTMGGIAAGIGTGTTALMATQQFQQLQAAVQDDLREVEKS

SEQ ID NO: 27 - protein, 53 aa, Friend murine leukemia virus <s300> Dai HY et al., Molecular cloning and characterization of an immunosuppressive and weakly oncogenic variant of friend murine leukemia virus, FIS-2 Journal of Virology 68 11 6976-6984 1994
EPVSLTLALLLGGLTMGGIAAGVGTGTTALVATQQFQQLHAAVQDDLKEVEKS

SEQ ID NO: 28 - protein, 53 aa, Murine leukemia virus <s300> Baylac-Kalabokias H et al., A new leukemogenic retrovirus isolated from tumor cells derived from a radio-induced lymphoma of C57BL/6 mice.  Analysis of the env and LTR sequences Virus Research 18 2 117-134 1991
EPVSLTLALLLGGLTMGGIAAGVGTGTTALVATQQFQQLQAAMHDDLKEVEKS

SEQ ID NO: 29 - protein, 27 aa, Ross River virus <s300> Dalgarno L et al., Ross River virus 26s RNA complete nucleotide sequence and deduced sequence of the encoded structural proteins Virology 129 1 170-187 1983
DYQCKVYTGVYPFMWGGAYCFCDSENT

SEQ ID NO: 30 - protein, 27 aa, Sindbis virus <s300> Strauss EG et al., Complete nucleotide sequence of the genomic RNA of Sindbis virus Virology 133 1 92-110 1984
DYTCKVFGGVYPFMWGGAQCFCDSENS

SEQ ID NO: 31 - protein, 19 aa, Measles virus <s300> Yeagle PL et al., Effects of the ′fusion peptide′ from measles virus on the structure of N-methyl dioleoylphosphatidylethanolamine membranes and their fusion with Sendai virus Biochimica et Biophysica Acta 1065 49-53 1991
FAGVVLAGAALGVAAAAQI

SEQ ID NO: 32 - protein, 19 aa, Measles virus <s300> Yeagle PL et al., Effects of the ′fusion peptide′ from measles virus on the structure of N-methyl dioleoylphosphatidylethanolamine membranes and their fusion with Sendai virus Biochimica et Biophysica Acta 1065 49-53 1991
FAGVVLAGAALGVATAAQI

SEQ ID NO: 33 - protein, 20 aa, Influenza virus <s300> Midoux et al., Specific gene transfer mediated by lactosylated poly-L-lysine into hepatoma cells Nucl. Acids Res. 21 871-78 1993
GLFGAIAGFIENGWEGMIDG

SEQ ID NO: 34 - protein, 20 aa, Influenza virus <s300> Midoux et al., Specific gene transfer mediated by lactosylated poly-L-lysine into hepatoma cells Nucl. Acids Res. 21 871-78 1993
GIFGAIAGFIENGWEGMIDG

SEQ ID NO: 35 - protein, 20 aa, Influenza virus <s300> Midoux et al., Specific gene transfer mediated by lactosylated poly-L-lysine into hepatoma cells Nucl. Acids Res. 21 871-78 1993
GLFGAIAGFIENGWEGMIDG

SEQ ID NO: 36 - protein, 20 aa, Influenza virus <s300> Midoux et al., Specific gene transfer mediated by lactosylated poly-L-lysine into hepatoma cells Nucl. Acids Res. 21 871-78 1993
GLFGAIAGFIEGGWEGMVDG

SEQ ID NO: 37 - protein, 20 aa, Influenza virus <s300> Midoux et al., Specific gene transfer mediated by lactosylated poly-L-lysine into hepatoma cells Nucl. Acids Res. 21 871-78 1993
GLFGAIAGFIEGGWEGLVDG

SEQ ID NO: 38 - protein, 20 aa, Influenza virus <s300> Midoux et al., Specific gene transfer mediated by lactosylated poly-L-lysine into hepatoma cells Nucl. Acids Res. 21 871-78 1993
GFFGAIAGFLEGGWEGMIAG